SEQUENCE LISTING (37 sequences)

SEQ ID NO: 1 - protein, 6 aa, Artificial Sequence
  Description of Artificial Sequence Synthetic peptide
TXGYGD

SEQ ID NO: 2 - protein, 5 aa, Artificial Sequence
  Description of Artificial Sequence Synthetic peptide
TVGXG

SEQ ID NO: 3 - DNA, 21 bp, Artificial Sequence
  Description of Artificial Sequence Synthetic oligonucleotide
aaggactatccttgaggcaaa

SEQ ID NO: 4 - DNA, 21 bp, Artificial Sequence
  Description of Artificial Sequence Synthetic oligonucleotide
atgggctaatatggatactaa

SEQ ID NO: 5 - DNA, 21 bp, Artificial Sequence
  Description of Artificial Sequence Synthetic oligonucleotide
aacatggatcctggatatgat

SEQ ID NO: 6 - DNA, 21 bp, Artificial Sequence
  Description of Artificial Sequence Synthetic oligonucleotide
gggaacgagtttagtatatta

SEQ ID NO: 7 - DNA, 21 bp, Artificial Sequence
  Description of Artificial Sequence Synthetic oligonucleotide
ctcctagtacttagaaataca

SEQ ID NO: 8 - DNA, 21 bp, Artificial Sequence
  Description of Artificial Sequence Synthetic oligonucleotide
ctggacgatcctctcagtgca

SEQ ID NO: 9 - DNA, 21 bp, Artificial Sequence
  Description of Artificial Sequence Synthetic oligonucleotide
caagctgctattccacattta

SEQ ID NO: 10 - DNA, 21 bp, Artificial Sequence
  Description of Artificial Sequence Synthetic oligonucleotide
tacgtgtatattcggcatcaa

SEQ ID NO: 11 - DNA, 21 bp, Artificial Sequence
  Description of Artificial Sequence Synthetic oligonucleotide
cggcgccagactggccgagta

SEQ ID NO: 12 - DNA, 21 bp, Artificial Sequence
  Description of Artificial Sequence Synthetic oligonucleotide
cgctctcgaggagtacactaa

SEQ ID NO: 13 - DNA, 21 bp, Artificial Sequence
  Description of Artificial Sequence Synthetic oligonucleotide
cttgatgaacttcatcgtaaa

SEQ ID NO: 14 - DNA, 21 bp, Artificial Sequence
  Description of Artificial Sequence Synthetic oligonucleotide
cccggaaagactgaaagaata

SEQ ID NO: 15 - DNA, 21 bp, Artificial Sequence
  Description of Artificial Sequence Synthetic oligonucleotide
atccatgaagctgatacgaca

SEQ ID NO: 16 - DNA, 21 bp, Artificial Sequence
  Description of Artificial Sequence Synthetic oligonucleotide
ctcgctgtacacaatgaccaa

SEQ ID NO: 17 - DNA, 21 bp, Artificial Sequence
  Description of Artificial Sequence Synthetic oligonucleotide
cggcatcaccttctacaacaa

SEQ ID NO: 18 - DNA, 21 bp, Artificial Sequence
  Description of Artificial Sequence Synthetic oligonucleotide
atggaggttgatggtaaggta

SEQ ID NO: 19 - DNA, 21 bp, Artificial Sequence
  Description of Artificial Sequence Synthetic oligonucleotide
gagcttgaatttgaaggtgta

SEQ ID NO: 20 - DNA, 21 bp, Artificial Sequence
  Description of Artificial Sequence Synthetic oligonucleotide
taaggtagagtcaattatgaa

SEQ ID NO: 21 - DNA, 21 bp, Artificial Sequence
  Description of Artificial Sequence Synthetic oligonucleotide
ctcgattactcaaatccctat

SEQ ID NO: 22 - DNA, 21 bp, Artificial Sequence
  Description of Artificial Sequence Synthetic oligonucleotide
cacggttaatgaagtctgcta

SEQ ID NO: 23 - DNA, 21 bp, Artificial Sequence
  Description of Artificial Sequence Synthetic oligonucleotide
accatgttaccctgtaattaa

SEQ ID NO: 24 - DNA, 21 bp, Artificial Sequence
  Description of Artificial Sequence Synthetic oligonucleotide
gaggatatgcttggtcgggta

SEQ ID NO: 25 - DNA, 21 bp, Artificial Sequence
  Description of Artificial Sequence Synthetic oligonucleotide
ctcgagcatctgcatacaata

SEQ ID NO: 26 - DNA, 21 bp, Artificial Sequence
  Description of Artificial Sequence Synthetic oligonucleotide
aagggagtaactcagattgat

SEQ ID NO: 27 - DNA, 21 bp, Artificial Sequence
  Description of Artificial Sequence Synthetic oligonucleotide
atgactttactctgactataa

SEQ ID NO: 28 - DNA, 21 bp, Artificial Sequence
  Description of Artificial Sequence Synthetic oligonucleotide
tccgttgggaatcatgcttat

SEQ ID NO: 29 - DNA, 21 bp, Artificial Sequence
  Description of Artificial Sequence Synthetic oligonucleotide
ccagttgggttctaaattgta

SEQ ID NO: 30 - DNA, 21 bp, Artificial Sequence
  Description of Artificial Sequence Synthetic oligonucleotide
ttccgtggatctgaattacaa

SEQ ID NO: 31 - DNA, 21 bp, Artificial Sequence
  Description of Artificial Sequence Synthetic oligonucleotide
tggcgccttatcaattgtcaa

SEQ ID NO: 32 - DNA, 21 bp, Artificial Sequence
  Description of Artificial Sequence Synthetic oligonucleotide
acggatctggatatacactat

SEQ ID NO: 33 - DNA, 21 bp, Artificial Sequence
  Description of Artificial Sequence Synthetic oligonucleotide
acgcttatgactcctaatgta

SEQ ID NO: 34 - DNA, 21 bp, Artificial Sequence
  Description of Artificial Sequence Synthetic oligonucleotide
caggaccaggtgaacaacaaa

SEQ ID NO: 35 - DNA, 21 bp, Artificial Sequence
  Description of Artificial Sequence Synthetic oligonucleotide
caccatgtctcgaatcaactt

SEQ ID NO: 36 - DNA, 21 bp, Artificial Sequence
  Description of Artificial Sequence Synthetic oligonucleotide
ctgcggctttgtccagttcaa

SEQ ID NO: 37 - DNA, 21 bp, Artificial Sequence
  Description of Artificial Sequence Synthetic oligonucleotide
caactcaatgacaaccgccaa

(19) United States
(12) Patent Application Publication
(10) Pub. No.: US 2011/0038852 A1
(43) Pub. Date: Feb. 17, 2011

(54) ANTIVIRALS THAT TARGET TRANSPORTERS, CARRIERS, AND ION CHANNELS

(75) Inventors: Eric MELDRUM, Zurich (CH); Stefan MOESE, Oberwil-Lieli (CH); Peder ZIPPERLEN, Urdorf (CH)

Correspondence Address: WILSON, SONSINI, GOODRICH & ROSATI, 650 PAGE MILL ROAD, PALO ALTO, CA, 94304-1050, US

(73) Assignee: 3-V BIOSCIENCES, INC., Menlo Park, CA (US)

(21) Appl. No.: 12/797,428

(22) Filed: Jun. 9, 2010

Related U.S. Application Data
(60) Provisional application No. 61/268,315, filed on Jun. 10, 2009.

(51) Int. Cl. A61K 39/395; A61K 31/7088; C12N 5/071; C12Q 1/70; A61P 31/12; A61P 31/16

(52) U.S. Cl. 424/130.1; 514/44 R; 435/375; 435/366; 435/5

(57) ABSTRACT

This invention provides methods for preventing or treating infection by viruses, in particular an influenza virus by modulating transporters, carriers, and ion channels. Methods to identify, validate, and classify the cellular proteins required by viruses during infection of host cells in order to select agents which can inhibit viral infection are described herein. The method employs a siRNA screening platform and uses gene silencing to map the ‘viral infectome’—a compilation of cellular proteins that the virus needs to establish infection and drive the infectious cycle. Charting the infectome provides information on the viral biology by the identification of host cell proteins involved in viral infection and allows the development of novel anti-viral drugs that prevent the viruses from establishing productive infection in cells.

### CROSS-REFERENCE

This application claims the benefit of U.S. Provisional Application No. 61/268,315, filed Jun. 10, 2009, which is incorporated herein by reference in its entirety.

### BACKGROUND OF THE INVENTION

Antiviral drugs are a class of medication used for the treatment of viral infections. Antiviral drugs are one class of antimicrobials, the larger group of which includes antibiotics, anti-fungals, and anti-parasitic drugs. Unlike antibacterial drugs, which can cover a wide range of pathogens, antiviral agents tend to be narrow in spectrum and have limited efficacy.

Common cold is a contagious respiratory illness caused by picornaviruses (including rhinoviruses) or coronaviruses. It is the most common infectious disease in humans and there is no known cure. Common symptoms include sore throat, runny nose, nasal congestion, and sneezing; sometimes accompanied by ‘pink eye’, muscle aches, fatigue, malaise, headaches, muscle weakness, uncontrollable shivering, loss of appetite, and rarely extreme exhaustion. Symptoms can be more severe in infants and young children. Although the disease is generally mild and self-limiting, patients with common colds often seek professional medical help, use over-the-counter drugs, and can miss school or work days. The annual cumulative societal cost of the common cold in developed countries is considerable in terms of money spent on remedies, and hours of lost productivity. There are no antiviral drugs approved to treat or cure the infection; all medications used are palliative and treat symptoms only. Alternative treatments such as vitamin C, echinacea, and zinc have been proposed but none of them have been shown to decrease the duration of the illness, and thus none of them are approved by the Food and Drug Administration or European Medicines Agency. Thus, there is a need for improved methods of inhibiting rhinoviral infections.

Instead of focusing on the virus itself and its proteins as a target, it is advantageous to develop a new generation of anti-viral drugs that interfere with host cell proteins involved in viral infection. By inhibiting the activity of these proteins it will be possible to inhibit the replication and production of progeny virus.

### SUMMARY OF THE INVENTION

In one aspect, a method of preventing or treating a viral infection is provided comprising administering to a subject in need thereof an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2. In one embodiment, the method comprises preventing a viral infection by administering to a subject in need thereof an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2. In one embodiment, the method comprises treating a viral infection by administering to a subject in need thereof an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2. In one embodiment, the infection can be a respiratory infection. In one embodiment, the virus can be a respiratory virus. In one embodiment, the virus can be a human rhinovirus. In one embodiment, the subject can be a human. In one embodiment, the agent can be an RNA, an antibody-based agent, or a small molecule.

In one aspect, a method of preventing or treating human rhinovirus infection is provided comprising administering to a subject in need thereof an agent that modulates a transporter, carrier, ion channel. In one embodiment, the transporter can be a V-ATPase, ATP-binding cassette (ABC) transporter, or Na+/K+-ATPase. In one embodiment, the ion channel can be a transient receptor potential (TRP) cation channel, voltage-gated potassium channel, or 5HT3-receptor. In one embodiment, the carrier can be a solute family carrier or APOA1. In one embodiment, the method comprises preventing human rhinovirus infection by administering to a subject in need thereof an agent that modulates a transporter, carrier, or ion channel. In one embodiment, the method comprises treating human rhinovirus infection by administering to a subject in need thereof an agent that modulates a transporter, carrier, or ion channel. In one embodiment, the subject can be a human. In one embodiment, the agent can be an RNA, an antibody-based agent, or a small molecule. In one embodiment, agent can be a transporter, carrier, or ion channel inhibitor.

In another aspect, a method of inhibiting infection of a cell by a virus is provided comprising contacting the cell with an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2. In one embodiment, method of inhibiting viral infection can be performed in vitro. In one embodiment, the method of inhibiting viral infection can be performed in vivo. In one embodiment, the infection can be a respiratory infection. In one embodiment, the virus can be a human rhinovirus. In one embodiment, the subject can be a human. In one embodiment, the agent can be an RNA, an antibody-based agent, or a small molecule. In one embodiment, the agent can be a transporter, carrier, or ion channel inhibitor.

In another aspect, a method of inhibiting infection of a cell by a human rhinovirus is provided comprising contacting the cell with an agent that modulates a transporter, carrier, or ion channel. In one embodiment, the method of inhibiting viral infection can be performed in vitro. In one embodiment, the method of inhibiting viral infection can be performed in vivo. In one embodiment, the subject can be a human. In one embodiment, the agent can be an RNA, an antibody-based agent, or a small molecule. In one embodiment, the transporter, carrier, or ion channel can be selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

In another aspect, a method is provided comprising: contacting a cell with an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2 with a virus, and determining whether the agent inhibits infection of the cell by the virus. In one embodiment, the infection can be a respiratory infection. In one embodiment, the virus can be a human rhinovirus. In one embodiment, the subject can be a human. In one embodiment, the agent can be an RNA, an antibody-based agent, or a small molecule.

In another aspect, a method is provided comprising contacting a cell with an agent that modulates a transporter, carrier, or ion channel with a human rhinovirus, and determining whether the agent inhibits infection of the cell by the human rhinovirus. In one embodiment, the subject can be a human. In one embodiment, the agent can be an RNA, an antibody-based agent, or a small molecule. In one embodiment, the agent can be a transporter, carrier, or ion channel inhibitor. In one embodiment, the transporter, carrier, or ion channel can be selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the structure of an HRV infection RNAi screen.

FIG. 2 depicts a typical R16-7 HRV staining pattern.

FIG. 3 is a Western blot of lysates of HeLa cells infected with rhinovirus (RV) serotypes 1A, 2, 14, 16, or 49 or mock-infected and blotted with monoclonal antibody R16-7. The antibody reacted with the viral capsid protein VP2 and with the VP2 precursors VP0 and P1, of both RV1A and RV16. This figure is taken from Mosser et al. J. Infectious Diseases 185, p 734, 2002.

FIG. 4 illustrates an experimental layout for an siRNA screen.

FIG. 5 illustrates a control layout for an siRNA screen.

FIG. 6 shows pictures that show a plate pipetted with 20 and 40 ul aliquots, respectively for an siRNA screen.

FIG. 7 illustrates results of an HRV infection study using siRNA against transporters, carriers, and ion channels.

FIGS. 8A-D illustrate chemical and structural formulas of groups that can form part of V-ATPase inhibitors.

FIGS. 9A-F illustrate chemical and structural formulas of groups that can form part of V-ATPase inhibitors.

### DETAILED DESCRIPTION OF THE INVENTION

The present invention provides compositions and methods for treatment of viral infections. In general, the compositions and methods for treatment of viral infections are directed toward modulating transporters, carriers, and ion channels. Transporters, carriers, and ion channels are involved in entry of various types of viruses into the host cells. The present invention embodies methods for treatment of viral infection, such as a human rhinovirus infection, that target transporters, carriers, and ion channels.

The methods of the invention include the identification of host cell genes that a virus uses for infection, replication and/or propagation. Also, described herein are methods of identifying agents that target specific host cell proteins, encoded by the identified host cell genes. Further, the present invention includes agents and methods for modulating the identified host cell targets. Such agents and methods are suitable for the treatment of viral infections. Such modulation of host cell targets can include either activation or inhibition of the host cell targets. Accordingly, compounds that modulate, e.g., inhibit, the activity of a non-viral protein, e.g., a host cell protein, e.g., a transporter, carrier, or ion channel, can be used as antiviral pharmaceutical agents.

In one embodiment the methods of the present invention can be used to develop antivirals to inhibit the infection of an animal subject, such as a human, by any of a plethora of viruses. In one embodiment the methods of the present invention are used to develop antivirals which inhibit the infection of a host by a respiratory virus. Respiratory viruses are most commonly transmitted by airborne droplets or nasal secretions and can lead to a wide spectrum of illness. Respiratory viruses include the respiratory syncytial virus (RSV), influenza viruses, coronaviruses such as SARS, adenoviruses, parainfluenza viruses and rhinoviruses (HRV).

### I. Viruses

In one embodiment host cell proteins are identified that a human rhinovirus (HRV) can use for infection or replication. The genus of rhinoviruses is a member of the Picornaviridae family of viruses. Genera within the family include the Genus Enterovirus, Rhinovirus, Cardiovirus, Aphthovirus, Hepatovirus, Parechovirus, Erbovirus, Kobuvirus, Teschovirus. Human rhinoviruses (HRV) include the most common viruses that infect humans and can cause the common cold. HRV are lytic in nature Rhinoviruses have single-stranded positive sense RNA genomes of between 7.2 and 8.5 kb in length. At the 5′ end of these genomes is a virus-encoded protein, and like mammalian mRNA, there is also a 3′ poly-A tail. The 5′-terminal UMP of the viral RNA is covalently linked to the small viral protein VPg (Paul A V, et al. Nature 1998, 393(6682):280-284). The 5′UTR contains two structural elements. One is the 5′-cloverleaf structure involved in the plus-strand RNA synthesis and in the process of switching from translation to replication (Huang H, et al. Biochemistry 2001, 40(27):8055-8064). The other is the internal ribosomal entry site (IRES) which promotes translation of the polyprotein. The 3′-UTR is necessary for efficient RNA replication, but the exact mechanism is still not well understood. In addition, species-specific internal cis-acting replication elements (cre) have been identified in human enteroviruses (HEV), HRV-A and HRV-B (Gerber K, Wimmer E, Paul A V, J Virol 2001, 75(22):10979-10990). The viral particles themselves are not enveloped and are icosahedral in structure. Rhinoviruses also grow best in temperatures between 33-35° C. They are also sensitive to acidic environment.

HRV viral proteins are transcribed as a single long polypeptide, which is cleaved into the viral structural and nonstructural proteins. Rhinoviruses are composed of a capsid that contains four viral proteins VP1, VP2, VP3 and VP4 (Rossmann M, et al. 1985 Nature 317 (6033): 145-53; Smith T, et al. 1986, Science 233 (4770): 1286-93). The isometric nucleocapsids are 22-40 nm in diameter. VP1, VP2, and VP3 form the major part of the protein capsid. The much smaller VP4 protein has a more extended structure and lies at interface between the capsid and the RNA genome. There are 60 copies of each of these proteins assembled as an icosahedron. Human antibodies that target epitopes lying on the exterior regions of VP1-VP3 play a role in the immune response to HRVs.

HRVs have two general modes of transmission: 1) via aerosols of respiratory droplets and 2) from contaminated surfaces, including direct person-to-person contact. The primary route of entry for rhinoviruses is the upper respiratory tract. Afterwards, an HRV binds to ICAM-1 (Inter-Cellular Adhesion Molecule 1) also known as CD54 (Cluster of Differentiation 54) receptors on respiratory epithelial cells. As the virus replicates and spreads, infected cells release chemokines and cytokines, which in turn activate inflammatory mediators. Infection occurs rapidly, with the rhinovirus adhering to surface receptors within 15 minutes of entering the respiratory tract. The incubation period is generally 8-10 hours before symptoms begin to occur. HRVs are the most frequent cause of infection across all age groups of the human population. Replication is often restricted to the upper respiratory tract leading to self-limited illnesses such as the common cold. However, HRV infections can also exacerbate pre-existing airway disorders, invade the lower respiratory tract and lead to serious complications.

HRV strains have been classified into more than 100 serologicaly distinct types based on the ability of a given serum to neutralize virus growth of a given strain in cell culture, although several serotypes share significant antigenic cross-reactivity (Nature 1967, 213(78):761-762). According to nucleotide sequence relatedness of some serotypes and to sequence comparison of all serotypes in the VP1 and VP4-VP2 capsid protein-coding regions, the serotypes generally segregate in two different groups: the HRV-A species and the HRV-B species. Molecular based genotyping has also revealed the existence of a third group, the HRV-C species (Lee W M et al. 2007 PLoS ONE 2(10): e966.doi:10.1371/journal.pone.0000966). In addition to the division of HRVs into three species, they have also been classified into major and minor groups according to receptor usage. The major group of HRVs binds ICAM1, whereas the minor group of viruses binds preferentially to LDL receptors (Greve J M, Davis et al. Cell 1989, 56(5):839-847). The existence of multiple serotypes within each of these two lineages and different receptor usage support the hypothesis of significant differences at the protein level. At least 99 out of 250 known serotypes of rhinoviruses affecting humans have been sequenced (Palmenberg, A. C. 2009 Science 324: 55). To date, there are no vaccines against these viruses as there is little-to-no cross-protection between serotypes.

Novel antiviral drugs have been developed for treating HRV infection. Interferon-alpha used intranasally was shown to be effective against rhinovirus infections. However, volunteers treated with this drug experienced some side effects, such as nasal bleeding, and began developing resistance to the drug. Subsequently, research into the treatment was abandoned. Pleconaril is an orally bioavailable antiviral drug being developed for the treatment of infections caused by picornaviruses (Pevear D, et al. 1999 Antimicrob Agents Chemother 43 (9): 2109-15). This drug acts by binding to a hydrophobic pocket in VP1 and stabilizes the protein capsid to such an extent that the virus cannot release its RNA genome into the target cell. When tested in volunteers, during the clinical trials, this drug caused a significant decrease in mucus secretions and illness-associated symptoms. Pleconaril is not currently available for treatment of rhinoviral infections, as its efficacy in treating these infections is under further evaluation (Fleischer R, Laessig K 2003 Clin Infect Dis 37 (12): 1722).

In another embodiment host cell proteins are identified that an influenza virus uses for infection or replication. Influenza viruses belong to Orthomyxoviridae family of viruses. This family also includes Thogoto viruses and Dhoriviruses. There are several types and subtypes of influenza viruses known, which infect humans and other species. Influenza type A viruses infect people, birds, pigs, horses, seals and other animals, but wild birds are the natural hosts for these viruses. Influenza type A viruses are divided into subtypes and named on the basis of two proteins on the surface of the virus: hemagglutinin (HA) and neuraminidase (NA). For example, an “H7N2 virus” designates an influenza A subtype that has an HA 7 protein and an NA 2 protein. Similarly an “H5N1” virus has an HA 5 protein and an NA 1 protein. There are 16 known HA subtypes and 9 known NA subtypes. Many different combinations of HA and NA proteins are possible. Only some influenza A subtypes (i.e., H1N1, H1N2, and H3N2) are currently in general circulation among people. Other subtypes are found most commonly in other animal species. For example, H7N7 and H3N8 viruses cause illness in horses, and H3N8 also has recently been shown to cause illness in dogs (see www.cdc.gov/flu/avian/gen-info/flu-viruses.htm).

Antiviral agents which target host cell proteins involved in influenza infection can be used to protect high-risk groups (hospital units, institutes caring for elderly, immuno-suppressed individuals), and on a case by case basis. A potential use for antiviral agents is to limit the spread and severity of the future pandemics whether caused by avian H5N1 or other strains of influenza virus. Avian influenza A viruses of the subtypes H5 and F17, including H5N1, H7N7, and H7N3 viruses, have been associated with high pathogenicity, and human infection with these viruses have ranged from mild (H7N3, H7N7) to severe and fatal disease (H7N7, H5N1). Human illness due to infection with low pathogenicity viruses has been documented, including very mild symptoms (e.g., conjunctivitis) to influenza-like illness. Examples of low pathogenicity viruses that have infected humans include H7N7, H9N2, and H7N2. (see www.cdc.gov/flu/avian/gen-info/flu-viruses.htm).

Influenza B viruses are usually found in humans but can also infect seals. Unlike influenza A viruses, these viruses are not classified according to subtype. Influenza B viruses can cause morbidity and mortality among humans, but in general are associated with less severe epidemics than influenza A viruses. Although influenza type B viruses can cause human epidemics, they have not caused pandemics. (see www.cdc.gov/flu/avian/gen-info/flu-viruses.htm).

Influenza type C viruses cause mild illness in humans and do not cause epidemics or pandemics. These viruses can also infect dogs and pigs. These viruses are not classified according to subtype. (see www.cdc.gov/flu/avian/gen-info/flu-viruses.htm).

Influenza viruses differ from each other in respect to cell surface receptor specificity and cell tropism, however they use common entry pathways. Charting these pathways and identification of host cell proteins involved in virus influenza transmission, entry, replication, biosynthesis, assembly, or exit allows the development of general agents against existing and emerging strains of influenza. The agents can also prove useful against unrelated viruses that use similar pathways. For example, the agents can protect airway epithelial cells against a number of different viruses in addition to influenza viruses.

In another embodiment host cell proteins are identified that an adenovirus or any viruses mentioned herein needs for infection or replication. Adenoviruses most commonly cause respiratory illness; symptoms of respiratory illness caused by adenovirus infection range from the common cold syndrome to pneumonia, croup, and bronchitis. Patients with compromised immune systems are especially susceptible to severe complications of adenovirus infection. Acute respiratory disease (ARD), first recognized among military recruits during World War II, can be caused by adenovirus infections during conditions of crowding and stress. Adenoviruses are medium-sized (90-100 nm), nonenveloped icosohedral viruses containing double-stranded DNA. There are 49 immunologically distinct types (6 subgenera: A through F) that can cause human infections. Adenoviruses are unusually stable to chemical or physical agents and adverse pH conditions, allowing for prolonged survival outside of the body. Some adenoviruses, such as AD2 and Ad5 (species C) use clathrin mediated endocytosis and macropinocytosis for infectious entry. Other adenoviruses, such as Ad3 (species B) use dynamin dependent endocytosis and macropinocytosis for infectious entry.

In another embodiment host cell proteins are identified that a respiratory syncytial virus (RSV) needs for infection or replication. RSV is the most common cause of bronchiolitis and pneumonia among infants and children under 1 year of age. Illness begins most frequently with fever, runny nose, cough, and sometimes wheezing. During their first RSV infection, between 25% and 40% of infants and young children have signs or symptoms of bronchiolitis or pneumonia, and 0.5% to 2% require hospitalization. Most children recover from illness in 8 to 15 days. The majority of children hospitalized for RSV infection are under 6 months of age. RSV also causes repeated infections throughout life, usually associated with moderate-to-severe cold-like symptoms; however, severe lower respiratory tract disease can occur at any age, especially among the elderly or among those with compromised cardiac, pulmonary, or immune systems. RSV is a negative-sense, enveloped RNA virus. The virion is variable in shape and size (average diameter of between 120 and 300 nm), is unstable in the environment (surviving only a few hours on environmental surfaces), and is readily inactivated with soap and water and disinfectants.

In another embodiment host cell proteins are identified that a human parainfluenza virus (HPIV) needs for infection or replication. HPIVs are second to respiratory syncytial virus (RSV) as a common cause of lower respiratory tract disease in young children. Similar to RSV, HPIVs can cause repeated infections throughout life, usually manifested by an upper respiratory tract illness (e.g., a cold and/or sore throat). HPIVs can also cause serious lower respiratory tract disease with repeat infection (e.g., pneumonia, bronchitis, and bronchiolitis), especially among the elderly, and among patients with compromised immune systems. Each of the four HPIVs has different clinical and epidemiologic features. The most distinctive clinical feature of HPIV-1 and HPIV-2 is croup (i.e., laryngotracheobronchitis); HPIV-1 is the leading cause of croup in children, whereas HPIV-2 is less frequently detected. Both HPIV-1 and -2 can cause other upper and lower respiratory tract illnesses. HPIV-3 is more often associated with bronchiolitis and pneumonia. HPIV-4 is infrequently detected, possibly because it is less likely to cause severe disease. The incubation period for HPIVs is generally from 1 to 7 days. HPIVs are negative-sense, single-stranded RNA viruses that possess fusion and hemagglutinin-neuraminidase glycoprotein “spikes” on their surface. There are four serotypes types of HPIV (1 through 4) and two subtypes (4a and 4b). The virion varies in size (average diameter between 150 and 300 nm) and shape, is unstable in the environment (surviving a few hours on environmental surfaces), and is readily inactivated with soap and water.

In another embodiment host cell proteins are identified that a coronavirus needs for infection or replication. Coronavirus is a genus of animal virus belonging to the family Coronaviridae. Coronaviruses are enveloped viruses with a positive-sense single-stranded RNA genome and a helical symmetry. The genomic size of coronaviruses ranges from approximately 16 to 31 kilobases, extraordinarily large for an RNA virus. The name “coronavirus” is derived from the Latin corona, meaning crown, as the virus envelope appears under electron microscopy to be crowned by a characteristic ring of small bulbous structures. This morphology is actually formed by the viral spike peplomers, which are proteins that populate the surface of the virus and determine host tropism. Coronaviruses are grouped in the order Nidovirales, named for the Latin nidus, meaning nest, as all viruses in this order produce a 3′ co-terminal nested set of subgenomic mRNA's during infection. Proteins that contribute to the overall structure of all coronaviruses are the spike, envelope, membrane and nucleocapsid. In the specific case of SARS a defined receptor-binding domain on S mediates the attachment of the virus to its cellular receptor, angiotensin-converting enzyme 2.

### II. Viral Infection Pathways

The host cell targets disclosed herein preferably play a role in the viral replication and/or infection pathways. Targeting of such host cell targets modulates the replication and/or infection pathways of the viruses. In preferred embodiments the identified host cell targets are directly or indirectly modulated with suitable agents. Such suitable agents can include small molecule therapeutics, protein therapeutics, or nucleic acid therapeutics. The modulation of such host cell targets can also be performed by targeting entities in the upstream or downstream signaling pathways of the host cell targets.

Like other viruses, the replication of HRV involves six phases; transmission, entry, replication, biosynthesis, assembly, and exit. Entry occurs by endocytosis, replication and vRNP assembly takes place in the nucleus, and the virus buds from the plasma membrane. In the infected patient, the virus targets airway epithelial cells. Preferably, in the methods described herein, at least one host cell target involved in such pathways is modulated.

The methods described herein are useful for development and/or identification of agents for the treatment of infections caused by the Abelson leukemia virus, Abelson murine leukemia virus, Abelson's virus, Acute laryngotracheobronchitis virus, Adelaide River virus, Adeno associated virus group, Adenovirus, African horse sickness virus, African swine fever virus, AIDS virus, Aleutian mink disease parvovirus, Alpharetrovirus, Alphavirus, ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian leukosis virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barmah Forest virus, Bebaru virus, Berrimah virus, Betaretrovirus, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, Buggy Creek virus, bullet shaped virus group, Bunyamwera virus supergroup, Bunyavirus, Burkitt's lymphoma virus, Bwamba Fever, CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, caprine arthritis virus, caprine encephalitis virus, Caprine Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, Charleville virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious eethyma virus, contagious pustular dermatitis virus, Coronavirus, Corriparta virus, coryza virus, cowpox virus, coxsackie virus, CPV (cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EIA virus, encephalitis virus, encephalomyocarditis group virus, encephalomyocarditis virus, Enterovirus, enzyme elevating virus, enzyme elevating virus (LDH), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumonitis virus, equine rhinovirus, Eubenangu virus, European elk papillomavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowlpox virus, Friend virus, Gammaretrovirus, GB hepatitis virus, GB virus, German measles virus, Getah virus, gibbon ape leukemia virus, glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, HCMV (human cytomegalovirus), hemadsorption virus 2, hemagglutinating virus of Japan, hemorrhagic fever virus, hendra virus, Henipaviruses, Hepadnavirus, hepatitis A virus, hepatitis B virus group, hepatitis C virus, hepatitis D virus, hepatitis delta virus, hepatitis E virus, hepatitis F virus, hepatitis G virus, hepatitis nonA nonB virus, hepatitis virus, hepatitis virus (nonhuman), hepatoencephalomyelitis reovirus 3, Hepatovirus, heron hepatitis B virus, herpes B virus, herpes simplex virus, herpes simplex virus 1, herpes simplex virus 2, herpesvirus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human B lymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immunodeficiency virus 1, human immunodeficiency virus 2, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I, human T lymphotropic virus II, human T lymphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kumba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MM virus, Mokola virus, Molluscipoxvirus, Molluscum contagiosum virus, monkey B virus, monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanirnavirus, Nariva virus, Ndumo virus, Neethling virus, Nelson Bay virus, neurotropic virus, New World Arenavirus, newborn pneumonitis virus, Newcastle disease virus, Nipah virus, noncytopathogenic virus, Norwalk virus, nuclear polyhedrosis virus (NPV), nipple neck virus, O'nyong'nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyam virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pestivirus, Phlebovirus, phocine distemper virus, Picodnavirus, Picornavirus, pig cytomegalovirus-pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect Hill virus, Provirus, pseudocowpox virus, pseudorabies virus, psittacinepox virus, quailpox virus, rabbit fibroma virus, rabbit kidney vaculolating virus, rabbit papillomavirus, rabies virus, raccoon parvovirus, raccoonpox virus, Ranikhet virus, rat cytomegalovirus, rat parvovirus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carpia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SARS virus, SDAV (sialodacryoadenitis virus), sealpox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV 1 virus group, STLV (simian T lymphotropic virus) type I, STLV (simian T lymphotropic virus) type II, STLV (simian T lymphotropic virus) type III, stomatitis papulosa virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalomyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, Una virus, Uukuniemi virus group, vaccinia virus, vacuolating virus, varicella zoster virus, Varicellovirus, Varicola virus, variola major virus, variola virus, Vasin Gishu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovirus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, and the Yug Bogdanovac virus. In one embodiment an infectome will be produced for each virus that includes an inventory of the host cellular genes involved in virus infection during a specific phase of viral infection, such cellular entry or the replication cycle.

For some viruses a great deal of progress has been made in the elucidation of the steps involved during infection of host cells. For example, experiments initiated in the early 1980s showed that influenza virus follows a stepwise, endocytic entry program with elements shared with other viruses such as alpha- and rhabdoviruses (Marsh and Helenius 1989; Whittaker 2006). The steps include: 1) Initial attachment to sialic acid containing glycoconjugates receptors on the cell surface; 2) signaling induced by the virus particle; 3) endocytosis by clathrin-dependent and clathrin-independent cellular mechanism; 4) acid-induced, hemaglutinin (HA)-mediated penetration from late endosomes; 5) acid-activated, M2 and matrix protein (M1) dependent uncoating of the capsid; and, 6) intra-cytosolic transport and nuclear import of vRNPs. These steps depend on assistance from the host cell in the form of sorting receptors, vesicle formation machinery, kinase-mediated regulation, organelle acidification, and, most likely, activities of the cytoskeleton.

Influenza attachment to the cells surface occurs via binding of the HAI subunit to cell surface glycoproteins and glycolipids that carry oligosaccharide moieties with terminal sialic acid residues (Skehel and Wiley 2000). The linkage by which the sialic acid is connected to the next saccharide contributes to species specificity. Avian strains including H₅N₁ prefer an a-(2,3)-link and human strains a-(2,6)-link (Matrosovich 2006). In epithelial cells, binding occurs preferentially to microvilli on the apical surface, and endocytosis occurs at base of these extensions (Matlin 1982). Whether receptor binding induces signals that prepare the cell for the invasion is not yet known, but it is likely because activation of protein kinase C and synthesis of phopshatidylinositol-3-phosphate (PI3P) are required for efficient entry (Sieczkarski et al. 2003; Whittaker 2006).

Endocytic internalization occurs within a few minutes after binding (Matlin 1982; Yoshimura and Ohnishi 1984). In tissue culture cells influenza virus makes use of three different types of cellular processes; 1) preexisting clathrin coated pits, 2) virus-induced clathrin coated pits, and 3) endocytosis in vesicles without visible coat (Matlin 1982; Sieczkarski and Whittaker 2002; Rust et al. 2004). Video microscopy using fluorescent viruses showed the virus particles undergoing actin-mediated rapid motion in the cell periphery followed by minus end-directed, microtubule-mediated transport to the perinuclear area of the cell. Live cell imaging indicated that the virus particles first entered a subpopulation of mobile, peripheral early endosomes that carry them deeper into the cytoplasm before penetration takes place (Lakadamyali et al. 2003; Rust et al. 2004). The endocytic process is regulated by protein and lipid kinases, the proteasome, as well as by Rabs and ubiquitin-dependent sorting factors (Khor et al. 2003; Whittaker 2006).

The membrane penetration step is mediated by low pH-mediated activation of the trimeric, metastable HA, and the conversion of this Type I viral fusion protein to a membrane fusion competent conformation (Maeda et al. 1981; White et al. 1982). This occurs about 16 min after internalization, and the pH threshold varies between strains in the 5.0-5.6 range. The target membrane is the limiting membrane of intermediate or late endosomes. The mechanism of fusion has been extensively studied (Kielian and Rey 2006). Further it was observed that fusion itself does not seem to require any host cell components except a lipid bilayer membrane and a functional acidification system (Maeda et al. 1981; White et al. 1982). The penetration step is inhibited by agents such as lysosomotropic weak bases, carboxylic ionophores, and proton pump inhibitors (Matlin 1982; Whittaker 2006).

To allow nuclear import of the incoming vRNPs, the capsid has to be disassembled. This step involves acidification of the viral interior through the amantadine-sensitive M2-channels causes dissociation of Mlfrom the vRNPs (Bukrinskaya et al. 1982; Martin and Helenius 1991; Pinto et al. 1992). Transport of the individual vRNPs to the nuclear pore complexes and transfer into the nucleus depends on cellular nuclear transport receptors (O'Neill et al. 1995; Cros et al. 2005). Replication of the viral RNAs (synthesis of positive and negative strands), and transcription occurs in complexes tightly associated with the chromatin in the nucleus. It is evident that, although many of the steps are catalyzed by the viral polymerase, cellular factors are involved including RNA polymerase activating factors, a chaperone HSP90, hCLE, and a human splicing factor UAP56. Viral gene expression is subject to complex cellular control at the transcriptional level, a control system dependent on cellular kinases (Whittaker 2006).

The final assembly of an influenza particle occurs during a budding process at the plasma membrane. In epithelial cells, budding occurs at the apical membrane domain only (Rodriguez-Boulan 1983). First, the progeny vRNPs are transported within the nucleoplasm to the nuclear envelope, then from the nucleus to the cytoplasm, and finally they accumulate in the cell periphery. Exit from the nucleus is dependent on viral protein NEP and M1, and a variety of cellular proteins including CRM1 (a nuclear export receptor), caspases, and possibly some nuclear protein chaperones. Phosphorylation plays a role in nuclear export by regulating M1 and NEP synthesis, and also through the MAPK/ERK system (Bui et al. 1996; Ludwig 2006). G protein and protein kinase signaling is involved in influenza virus budding from infected host cells (Hui E. and Nayak D, 2002).

The three membrane proteins of the virus are synthesized, folded and assembled into oligomers in the ER (Doms et al. 1993). They pass through the Golgi complex; undergo maturation through modification of their carbohydrate moieties and proteolytic cleavage. After reaching the plasma membrane they associate with M1 and the vRNPs in a budding process that results in the inclusion of all eight vRNPs and exclusion of most host cell components except lipids.

Influenza infection is associated with activation of several signaling cascades including the MAPK pathway (ERK, JNK, p38 and BMK-1/ERK5), the IkB/NF-kB signaling module, the Raf/MEK/ERK cascade, and programmed cell death (Ludwig 2006). These result in a variety of effects that limit the progress of infection such as transcriptional activation of IFNb, apoptotic cell death, and a block in virus escape of from late endosomes (Ludwig 2006).

Most previous studies on virus-cell interactions were performed in tissue culture using tissue culture- or egg-adapted virus strains. The viruses in these examples were adapted in such as manner that changes were induced that affected receptor binding and tropism (Matrosovich 2006). Infection with wild-type pathogenic strains is provides a more natural picture of viral interaction with host proteins. It is known that in the human airways influenza A and B primarily infect non ciliated epithelial cells in the upper respiratory track carrying NeuSAc a-(2,6)-Gal, whereas avian strains infect ciliated epithelial cell with a-(2,3)-linked sialic acids deeper in the airways (Matrosovich et al. 2004a).

Additionally, progress has been made in the elucidation of the steps involved during infection by HRV of host cells. Selected events in rhinovirus infection of the normal human airway can be regarded as occurring sequentially. Initial steps in rhinovirus pathogenesis are believed to include viral entry through the nose, mucociliary transport of virus to the posterior pharynx, and initiation of infection in ciliated and non-ciliated epithelial cells of the upper airway. Viral replication peaks on average within 48 h of initiation of infection and persists for up to 3 wk. Infection is followed by activation of several inflammatory mechanisms, which can include release or generation of interleukins, bradykinins, prostaglandins, and possibly histamine and stimulation of parasympathetic reflexes. Pathophysiologic processes are initiated, which include vasodilatation of nasal blood vessels, transudation of plasma, glandular secretion, and stimulation of nerve fibers, causing pain and triggering sneeze and cough reflexes. The resultant clinical illness is a rhinosinusitis, pharyngitis, and bronchitis, which, on average, lasts 1 wk.

Changes in gene expression profiles during in vivo rhinovirus infections have been identified (Proud D. et al. Am J Respir Crit. Care Med Vol 178. pp 962-968, 2008). Nasal epithelial scrapings were obtained before and during experimental rhinovirus infection, and gene expression was evaluated by microarray. Viperin is identified as an antiviral protein induced by interferon (IFN), viral infections, and pathogen-associated molecules. Naturally acquired rhinovirus infections, cultured human epithelial cells, and short interfering RNA knockdown were used to further evaluate the role of viperin in rhinovirus infections. Symptom scores and viral titers were measured in subjects inoculated with rhinovirus or a sham control, and changes in gene expression were assessed 8 and 48 hours after inoculation Rhinovirus-induced changes in gene expression were not observed 8 hours after viral infection, but 11,887 gene transcripts were significantly altered in scrapings obtained 2 days post-inoculation. Major groups of up-regulated genes include chemokines, signaling molecules, interferon-responsive genes, and antivirals. Rhinovirus infection significantly alters the expression of many genes associated with the immune response, including chemokines and antivirals. Some of the genes markedly induced by HRV-16 infection include but are not limited to CCL2, CCL8, CXCL11, CXCL10, CXCL13, CXCL9, CCL20, IFIT2, GBP1, IFIT1, GIP2, IFIT4, IL28B, IRF7, CIG5, NOS2A, OAS3, OASL, OAS2, OAS1, MX2, MX1, PLSCR1, SOCS1, SOCS2, MDA5, RIGI, SOCS3, ICAM-1, HAPLN3, MMP12, EPSTI1, and TNC.

### III. Transporters, Carriers, and Ion Channels

One aspect of the invention is compositions and methods that modulate the activity of transporters, carriers, and ion channels to treat a viral infection.

A. Transporters

1. Vacuolar-ATPases

One aspect of the invention is compositions and methods that modulate the activity of vacuolar (V-type) ATPases (or vacuolar H⁺-ATPases) to treat or prevent a viral infection. V-ATPases are ATP-dependent proton pumps that can transport protons into intracellular organelles. They can be found in the plasma membranes of osteoclasts, renal intercalated cells, and macrophages. V-ATPases are present on ubiquitous intracellular acidic compartments such as lysosomes, endosomes, Golgi apparatus and secretory vesicles. V-ATPases play roles in energy conservation, secondary active transport, acidification of intracellular compartments, zymogen activation, prohormone processing, protein degradation, receptor-mediated endocytosis, neurotransmitter uptake, synaptic vesicle proton gradient generation, and cellular pH homeostasis.

V-ATPases play roles in the entry of viruses and toxins into cells. Enveloped viruses, such as influenza virus, enter cells via acidic endosomal compartments. The low pH generated by V-ATPases causes the viral coat to fuse with the endosomal membrane, resulting in the formation of a membrane pore and the release of the viral mRNA molecules into the cytoplasm. For example, hemaggultinin (HA)² coat protein of influenza can mediate fusion of the viral membrane and the endosomal membrane after endosomal uptake of the viral particle. Release of the viral nucleic acid into the cytoplasm of the host cell plays a role in viral replication. Furthermore, the acidic environment can induce toxins that enter the cell via endocytosis to enter the cytoplasm.

V-type ATPases are composed for 14 different subunits. These subunits are organized into a transmembrane proton-conducting sector (V0) and an extramembrane catalytic sector (V1). The V0 domain consists of five different subunits, a, c, c′, c″, and d, in a stoichiometry of adc′c″c₄. The catalytic V1 domain contains eight different subunits (A-H), some of which are present in multiple copies. The V1 domain contains 3 copies each of the A and B subunits, 2 copies of the G subunit, 1 or 2 copies of subunit H and single copies of the remaining subunits. The nucleotide binding sites of V-ATPase appear to be at the interface between the A and B subunits. Most of the residues that constitute the ATP-binding interface are contributed by the A subunits. Subunit A plays a role in ATP hydrolysis, and subunit B contains an ATP binding site and can play a regulatory role.

The compositions and methods of the present invention can modulate the processes that regulate V-ATPase activity. In response to various stimuli, V-ATPase complexes can reversibly dissociate into their component V1 and V0 domains, thereby shutting down ATP-dependent proton transport. This occurs in dendritic cells upon activation of antigen processing. In low glucose, V-ATPase disassembly occurs, resulting in separated V1 and V0. In yeast, reassembly can be mediated by RAVE (Rav1p, Rav2p, Skp1p). In addition, the glycolytic enzyme aldolase mediates V-ATPase assembly and activity by physical association with the proton pump. Reversible disulfide bond formation between residues in subunit A can lock the catalytic site into a conformation that is unable to undergo ATP hydrolysis. Finally, the actin cytoskeleton plays roles in regulating the density of V-ATPases at the plasma membrane. Compositions and methods of the provided invention can modulate the activity of the V-ATPase components ATP6AP2, ATP6V1A, ATP6V1B2, or ATP6V1C1 to treat or prevent a viral infection.

a. ATP6AP2

In one embodiment, the provided invention includes compositions and methods that modulate the activity of ATP6AP2 to treat or prevent a viral infection. ATP6AP2 (also known as ATPase, H+ transporting, lysosomal accessory protein 2, 5730403E06Rik, APT6M8-9, ATP61P2, ATP6M8-9, ELDF10, HT028, M8-9, MGC94783, MGC99577, MRXE, MSTP009, N14F, Prorenin/Renin Receptor, PSEC0072, RENIN RECEPTOR, and XMRE) encodes a protein that can associate with the transmembrane sector of the V-type ATPases.

b. ATP6V1A

In another embodiment, the provided invention includes compositions and methods that modulate the activity of ATP6V1A to treat or prevent a viral infection. ATP6V1A (also known as ATPase, H+ transporting, lysosomal 70 kDa, VI subunit A; A1647066, ATP6A1, Atp6a2, ATP6V1A1, HO68, LOC685232, VA68, Vaa1, Vma1, and VPP2) encodes a protein that is one of two V1 domain A subunit isoforms and is found in all tissues.

c. ATP6V1B2

In another embodiment, the provided invention includes compositions and methods that modulate the activity of ATP6V1B2 to treat or prevent a viral infection. ATP6V1B2 (also known as ATPase, H+ transporting, lysosomal 56/58 kDa, VI subunit B2, AI194269, AI790362, ATP6B1B2, ATP6B2, Atpase H+ Transporting Lysosomal Isoform 2, HO57, R74844, V-ATPASE B2, VATB, Vma2, VPP3) encodes one of two V1 domain B subunit isoforms and is the only B isoform highly expressed in osteoclasts.

d. ATP6V1C1

In another embodiment, the provided invention includes compositions and methods that modulate the activity of ATP6V1C1 to treat or prevent a viral infection. ATP6V1C1 (also known as ATPase, H+ transporting, lysosomal 42 kDa, V1 subunit C1; 1700025B18Rik, ATP6C, ATP6D, FLJ20057, MGC109315, MGC140015, U13839, V-Atpase c, VATC, and Vma5) is one of two genes that encode the V1 domain C subunit proteins and is found ubiquitously. This C subunit is analogous but not homologous to gamma subunit of F-ATPases.

2. ATP-Binding Cassette (ABC) Transporters

In another embodiment, the provided invention includes compositions and methods that modulate the activity of an ATP-binding cassette (ABC) transporter to treat or prevent a viral infection. The ATP-binding cassette (ABC) transporter superfamily contains membrane proteins that can bind to ATP and use the energy to translocate a variety of substrates, for example, metabolic products, lipids and sterols, and drugs, across extra- and intracellular membranes (reviewed in Dean M. (2002) The Human ATP-Binding Cassette (ABC) Transporter Superfamily http://www.ncbi.nlm.nih.gov/bookshelf/br.fcgi?book=mono_{—}001&part=A137#A139). ABC transporters contain cytoplasmic ATP-binding domain(s), or nucleotide-binding folds (NBFs), that contain Walker A and B motifs separated by approximately 90-120 amino acids. ABC genes also contain a signature (C) motif N-terminal of the Walker B site. Functional ABC transporters typically contain two NBFs and two TM domains. The TM domains contain 6-11 membrane-spanning α-helices that can contribute to substrate specificity. In eukaryotes, ABC transporters can move compounds from the cytoplasm to the outside of the cell or into an intracellular compartment (endoplasmic reticulum (ER), mitochondria, peroxisome).

There are at least 48 known human ABC transporters, and these can be divided in seven distinct subfamilies based on similarity in gene structure, domain order, and sequence homology in the NBF and TM domains. The seven mammalian ABC gene subfamilies include ABCA (ABC1), ABCB (MDR/TAP), ABCC(CFTR/MRP), ABCD (ALD), ABCE (OABP), ABCF (GCN20), and ABCG (White). Compositions and methods of the provided invention can modulate the activity of the ABC transporters ABCC4, ABCE1, and TAP2 to treat or prevent a viral infection.

a. ABCC4

In another embodiment, the provided invention includes compositions and methods that modulate the activity of ABCC4 to treat or prevent a viral infection. ABCC4 is also known as ATP-binding cassette, sub-family C (CFTR/MRP), member 4; D630049P08Rik, EST170205, MOAT-B, MOATB, and MRP4. This protein is a member of the ABCC (CFTR/MRP) subfamily of ABC transporters which is involved in multi-drug resistance. This protein can play a role in cellular detoxification as a pump for its substrate, organic anions.

There are 11 other members of the ABCC subfamily with a diverse functional spectrum that includes ion transport, cell-surface receptor, and toxin secretion activities. The ABCC4, ABCC5, ABCC11, and ABCC12 proteins lack an N-terminal domain that is not essential for transport function. The ABCC4 and ABCC5 proteins can confer resistance to nucleosides including PMEA and purine analogs. The CFTR protein is a chloride ion channel that can play a role in exocrine secretions (mutations in CFTR cause cystic fibrosis). ABCC8 and ABCC9 proteins can bind sulfonylurea and regulate potassium channels involved in modulating insulin secretion. ABCC1, ABCC2, and ABCC3 can transport drug conjugates to glutathionine and other organic anions.

b. ABCE1

In another embodiment, the provided invention includes compositions and methods that modulate the activity of ABCE1 to treat or prevent a viral infection. ABCE1 is also known as ATP-binding cassette, sub-family E (OABP), member 1; ABC38, Atp-binding cassette subfamily e member 1a, C79080, HP68, HuHP68, OABP, RI, RLI, RNASEL1, RNASELI, RNS41, and RNS4I. This protein is the sole member of the ABCE (OABP) subfamily and lacks a TM domain. Alternatively referred to as the RNase L inhibitor, this protein can block the activity of ribonuclease L. Activation of ribonuclease L leads to inhibition of protein synthesis in the 2-5A/RNase L system, the central pathway for viral interferon action.

c. TAP2

In another embodiment, the provided invention includes compositions and methods that modulate the activity of TAP2 to treat or prevent a viral infection. TAP2 is also known as transporter 2, ATP-binding cassette, sub-family B (MDR/TAP); ABC18, ABCB3, AI462429, Antigen Peptide Transporter 2, APT2, Cim, D6S217E, Ham-2, jas, MGC108646, MTP2, PSF2, RING11, and Y1. This protein is a member of the ABCB (MDR/TAP) subfamily. Members of the ABCB (MDR/TAP) subfamily are involved in multidrug resistance. The protein encoded by this gene is involved in antigen presentation. This protein can form a heterodimer with ABCB2 to transport peptides from the cytoplasm to the endoplasmic reticulum. Mutations in this gene can be associated with ankylosing spondylitis, insulin-dependent diabetes mellitus, and celiac disease. Alternative splicing of this gene can produce two products which differ in peptide selectivity and level of restoration of surface expression of MHC class 1 molecules.

The ABCB subfamily contains both full transporters and half transporters. ABCB1 (MDR/PGY1) can confer a MDR phenotype to cancer cells and can function in the blood-brain barrier and the liver. The ABCB4 and ABCB11 proteins can play a role in the secretion of bile acids. The ABCB9 half transporter can localize to lysosomes. ABCB6, ABCB7, ABCB8, and ABCB10 can localize to the mitochondria and play roles in iron metabolism and transport of Fe/S protein precursors.

3. Na⁺/K⁺-ATPases

In another aspect, the present invention provides compositions and methods that modulate the activity of a Na⁺/K⁺-ATPase (NKA) to treat or prevent a viral infection. NKAs are a subfamily of the P-Type cation transport ATPases and are integral membrane proteins composed of two differently sized subunits. The smaller glycoprotein, termed beta, is required for the insertion of NKA into the plasma membrane. There are four beta subunits: ATP1B1, ATP1B2, ATP1B3, and ATP1B4. The large catalytic subunit, termed alpha, is the catalytic subunit. There are four alpha subunits: ATP1A1, ATP1A2, ATP1A3, and ATP1A4. NKAs can exist as asymmetric tetramers. NKA uses the energy from ATP hydrolysis to transport Na⁺ out of a cell and K⁺ into a cell. The establishment of Na⁺ and K⁺ ion gradients is important for osmoregulation, sodium coupled transport organic and inorganic molecules, and the electrical excitability of nerve and muscle.

Another aspect of the present invention provides compositions and methods that modulate one or more components of NKA signaling pathways to treat a viral infection. NKA can act as a signal transducer independently of its function as an ion pump. One means by which NKA can be a signal transducer is by binding the cardiotonic steroid ouabain. Binding of NKA to ouabain can result in a change in conformation in NKA. This allosteric effect is transferred to the NKA binding partner 1,4,5-triphosphate receptor (IP3R), a tetrametric calcium channel that is inserted in the membrane of the endoplasmic reticulum (ER). This interaction can result in a rhythmic opening and closure of the calcium channel. The ouabain dependent calcium oscillations can specifically activate the calcium-dependent transcription factor NF-kB, which can result in cell proliferation and can have an anti-apoptotic effect.

An alternative pathway for NKA signaling involves activation of the Src and EGF receptors. When ouabain binds NKA, Src is phosphorylated. Signal cascades, including MAPK signaling pathways, are activated, which can have both growth promoting and antioxidant effects.

The kinases PKA and PKC can phosphorylate the α-subunit of NKA. The α-subunit can also be phosphorylated by a tyrosine kinase.

NKA can interact with other proteins, including phosphoinositide-3 kinase, AP-2, and ankyrin. The α-subunit of Na⁺/K⁺-ATPase can interact with cofilin (Lee K. et al. (2001) Biochem J. 353:377-385).

ATP1A1

In another embodiment, the provided invention includes compositions and methods that modulate the activity of ATP1A1 to treat or prevent a viral infection. ATP1A1 is also known as ATPase, Na+/K+ transporting, alpha 1 polypeptide; Atpa-1, BC010319, MGC3285, MGC38419, MGC51750, Na K alpha 1, Na+/K+ atpase alpha1, NA,K-ATPASE ALPHA SUBUNIT 1, NKA ALPHA 1, and Nkaa1b. This gene encodes an alpha 1 subunit.

B. Carriers

1. Solute Carrier Family

In another embodiment, the provided invention includes compositions and methods that modulate the activity of a solute carrier family member to treat or prevent a viral infection. The SoLute Carrier (SLC) group of membrane transport proteins include over 300 members organized into 47 families. Solutes transported by the various SLC group members include both charged and uncharged organic molecules and inorganic ions. SLCs contain a number of hydrophobic transmembrane alpha helices connected to each other by hydrophilic intra- or extra-cellular loops. Depending on the SLC, these transporters can function as either monomers or obligate homo- or hetero-oligomers. The SLC group include facilitative transporters (allow solutes to flow with their electrochemical gradients) and secondary active transporters (allow solutes to flow against their electrochemical gradient by coupling to transport of a second solute that flows with its gradient such that the overall free energy change is still favorable). Members of the SLC group can be located in the outer cell membrane, but some members are located in mitochondria or other intracellular organelles.

By convention of the nomenclature system, members within an individual SLC family have greater than 20-25% sequence homology to each other. Names of individual SLC members have the following format: SLCnXm, where: SLC is the root name (SoLute Carrier), n=an integer representing a family (e.g., 1-47), X=a single letter (A, B, C, . . . ) denoting a subfamily, and m=an integer representing an individual family member (isoform).

a. SLC35C2

In another embodiment, the provided invention includes compositions and methods that modulate the activity of SLC35C2 to treat or prevent a viral infection. SLC35C2 is also known as solute carrier family 35, member C2, BA394O2.1, C20orf5, C85957, CGI-15, D2Wsu58e, FLJ37039, FLJ46434, LOC100128167, MGC18664, MGC20633, MGC32079, MGC39183, and OVCOV1. Oxygenation levels play an important role in the regulation of cellular invasiveness which occurs during early implantation when the trophoblast cells invade the uterus as well as during tumour progression and metastasis. This gene, which is regulated by oxygen tension, is induced in hypoxic trophoblast cells and is overexpressed in ovarian cancer. Two protein isoforms are encoded by transcript variants of this gene.

b. SLC7A1

In another embodiment, the provided invention includes compositions and methods that modulate the activity of SLC7A1 to treat or prevent a viral infection. SLC7A1 (also known as 4831426K01RIK, AI447493, ATRC1, CAT-1, EcoR, ER, ERR, HCAT1, mCAT-1, Rec-1, REC1L, REV-1, and CAT1) can act as a cationic amino acid transporter and as an ecotropic retrovirus receptor. SLC7A1 can play a role in the Na⁺-independent y⁺ cationic amino acid transport system. SLC7A1 mediated transport can be downregulated on protein kinase C(PKC) activation (Gräf P et al. (2001) Br J Pharmacol 132:1193-1200).

2. APOA1

In another embodiment, the provided invention includes compositions and methods that modulate the activity of APOA1 to treat a viral infection. APOA1 is also known as Ai, ALP-1, Apola, APOA-I, APOLIPOPROTEIN A-I, APOLIPOPROTEIN A-I G1, APOLIPOPROTEIN A1, Brp-14, HDLA1, LP(A-I), Ltw-1, Lvtw-1, MGC102525, MGC117399, Sep-1, and Sep-2. This gene encodes apolipoprotein A-I, which is the major protein component of high density lipoprotein (HDL) in plasma. APOA1 acts as a carrier for HDL cholesterol. The protein promotes cholesterol efflux from tissues to the liver for excretion, and it is a cofactor for lecithin cholesterolacyltransferase (LCAT) which is responsible for the formation of most plasma cholesteryl esters. This gene is closely linked with two other apolipoprotein genes on chromosome 11. Defects in this gene are associated with HDL deficiencies, including Tangier disease, and with systemic non-neuropathic amyloidosis.

C. Ion channels

1.5-11T3 Receptor

In another aspect, the present invention provides compositions and methods that modulate the activity of 5-HT3 receptor to treat or prevent a viral infection. The 5-HT3 (5-hydroxytryptamine-3) receptor is a ligand-gated ion channel. The receptor contains 5 subunits that are positioned around a central ion conducting pore. The subunits are proteins encoded by the genes HTR3A, HTR3B, HTR3C, HTR3D, and/or HTR3E. Functional channels can be comprised of five identical 5-HT3A subunits (homopentameric) or a mixture of 5-HT3A and one of the other four subunits (5-HT3B, 5-HT3C, 5-HT3D, or 5-HT3E; heteropentameric). The pore of the 5-HT3 channel is permeable to sodium, potassium, and calcium ions. The 5-HT3 receptor can bind serotonin (5-hydroxytryptamine, or 5-HT), a biogenic hormone that functions as a neurotransmitter, a hormone, and a mitogen.

5-HT3 receptors are expressed throughout the central and peripheral nervous systems and mediate a variety of physiological functions. The HTR3A, HTR3B, and HTR3C genes are expressed in the CNS and periphery; HTR3D, and HTR3E are expressed in the GI tract. Activation of the 5-HT3 receptor can modulate activities including, for example, drug-induced emesis and nociception, gut motility, peristalsis, visceral sensation, and secretion. Postsynaptic 5-HT3 receptors can mediate fast excitatory synaptic transmission in rat neocortical interneurons and amygdala, and in ferret visual cortex. 5-HT3 receptors are also present on presynaptic nerve terminals, where they are thought to mediate or modulate neurotransmitter release. 5-HT3 receptors can be found at the ends of afferent branches of the vagus nerve. The vagus nerve sends signals to the vomit center of the brain in the medulla oblongata and in the chemoreceptor trigger zone (CTZ) of the brain. This receptor causes fast, depolarizing responses in neurons after activation.

HTR3A

In another embodiment, the provided invention includes compositions and methods that modulate the activity of HTR3A to treat or prevent a viral infection. HTR3A is also known as 5-hydroxytryptamine (serotonin) receptor 3A, 5-HT-3, 5-HT3A, 5-HT3R, 5HT3A RECEPTOR, HTR3.

2. Transient Receptor Potential (TRP Cation Channels)

In another aspect, the present invention provides compositions and methods that modulate the activity of transient receptor potential (TRP cation channels) to treat or prevent a viral infection. Transient receptor potential (TRP) cation channels are diverse members of a family of ion channels that are permeable to cations, including sodium, calcium and magnesium. Most TRP channels contain 6 membrane-spanning helices with intracellular N- and C-termini. Mammalian TRP channels are activated and regulated by a wide variety of stimuli. They play roles in cellular and sensory systems including photosensation, osmosensation, thermosensation, and taste sensation.

There are seven subfamilies in the TRP superfamily that are divided into two groups: Group 1 subfamilies include TRPC, TRPV, TRPM, TRPA, and TRPN. Group 2 subfamilies include TRPP and TRPML. The mammalian TRP mucolipin (TRPML) subfamily consists of three members: TRPML1, TRPML2, and TRPML3 (MCOLN3). Each TRPML protein can form homo- and heteromultimers with other members of the TRPML subfamily.

MCOLN3

In another embodiment, the provided invention includes compositions and methods that modulate the activity of MCOLN3 to treat a viral infection. MCOLN3 is also known as 6720490021Rik, FLJ11006, FLJ36629, MGC124245, MGC124246, MGC71509, TRP-ML3, TRPML3, and Va. Mucolipins constitute a family of cation channel proteins with homologs in mouse, Drosophila, and C. elegans. Mutations in the human MCOLN1 gene (MIM 605248) cause mucolipodosis IV (MIM 262650). Mutations in MCOLN3 can result in hear loss and pigmentation defects associated with vaitint-waddler mice. The subcellular distribution of MCOLN3 is influenced by TRPML1 or TRPML2. When expressed individually, TRPML1 and TRPML2 are lysosomal membrane proteins whereas MCOLN3 can be in the ER. When MCOLN3 is coexpressed with TRPML1 or TRPML2, it can translocate to the lysosomes (Venkatachalam et al. (2006). J Biol. Chem. 281:17517-17527).

3. Voltage-Gated Potassium Channels

Voltage-gated potassium (Kᵥ) channels are ion channels that open or close in response to changes in the transmembrane voltage. They are tetrameric channels with each α-subunit containing a voltage sensor and contributing to the central pore. There are a number of different α-subunits, and Kᵥ channels can be homotetramers or heterotetramers of α-subunits. Kᵥ channels contain pore-lining P-loops which have a consensus amino acid sequence, -TXGYGD-, called the K⁺-channel “signature sequence”. The Kᵥ channel α-subunit contains six transmembrane regions (TM3; S1-S6). Both N and C termini are on the intracellular side of the membrane. The S4 segment of Kᵥ channels contains positively charged amino acids (Arg or Lys) at every third position and is part of the voltage sensor responsible for voltage-dependent gating. Voltage-gated K⁺ channels are selective for K⁺ over other cations such as Na⁺. There is a selectivity filter at the narrowest part of the transmembrane pore. As K⁺ passes through the pore, interactions between potassium ions and water molecules are prevented and the K⁺ interacts with the Thr-Val-Gly-X-Gly sequences from the four channel subunits.

Based on sequence homology of the hydrophobic transmembrane cores, the alpha subunits of voltage-gated potassium channels have been grouped into 12 classes labeled Kᵥ1-12. The alpha subunits can be placed into groups including: 1) Delayed rectifier (slowly inactivating or non-inactivating), 2) A-type potassium channel (rapidly inactivating) 3) Outward-rectifying, 4) Inward-rectifying, 5), Slowly activating, and 6) Modifier/silencer, which do not form functional channels as homotetramers but can heterotetramerize with Kᵥα2 family members to form conductive channels. Four sequence-related potassium channel genes—shaker, shaw, shab, and shal—have been identified in Drosophila, and each has a human homolog(s).

Beta subunits are auxiliary proteins which can associate with alpha subunits in a α4β4 stoichiometry. These subunits can modulate the activity of Kᵥ channels, but they do not conduct current on their own.

Many Kᵥ channels can be subjected to post-translational modification, including ubiquitinylation, phosphorylation, and palmitoylation.

Kᵥ channels have diverse functions include regulating neurotransmitter release, heart rate, insulin secretion, neuronal excitability, epithelial electrolyte transport, smooth muscle contraction, and cell volume.

KCNB2

In another embodiment, the provided invention includes compositions and methods that modulate the activity of KCNB2 to treat a viral infection. KCNB2 is also known as potassium voltage-gated channel, Shab-related subfamily, member 2; 9630047L19RIK, BB130875, and KV2.2. This gene encodes a member of the potassium channel, voltage-gated, shab-related subfamily. This member is a delayed rectifier potassium channel. The gene is expressed in gastrointestinal smooth muscle cells. KCNB2 functions in maintaining membrane potential and modulating electrical excitability in neurons. The angiotensin II type 1 receptor can play a role in inhibition of KCNB2 in the brainstem and in hypothalamic neurons (Gelband et al. (1999) Circ Res 84:352-359).

Compositions of the present invention can include small molecules, antibody-based agents, protein therapeutics, and nucleotide therapeutics. Nucleotide therapeutics can include shRNA, siRNA, miRNA, antisense RNA, ribozymes, aptamers, and restriction enzymes.

IV. Methods and Apparatus for Identification of Host Cell Proteins that Play a Role in Viral Infection.

Cell invasion and productive infection by viruses involves a step-wise program where a few of the events are mediated by viral proteins and enzymes, but the rest depends on cellular functions. To obtain a complete inventory of the cellular proteins involved, embodiments utilizing a systems biology approach are quite useful. Embodiments involving the systematic identification of essential genes involved in HRV infection in tissue culture cells provide an informative avenue of discovery. Systems biology approaches involving genome-wide libraries of siRNAs, and high-throughput instrument platforms can quickly and efficiently identify host cell proteins involved in viral infection from a plethora of candidate proteins.

In one embodiment systematic identification of host cell proteins is performed with the use of an automated high-throughput siRNA screening technology combined with the genomic data base information. Wherein, the genomic database can be derived from any species for whose genomic sequence is known, including the human, the mouse, or an avian species. In another embodiment a screening platform with advanced robotics and screening technology with such as the RNAi Image-based Screening Center' (RISC), can be used. The siRNA screening can be practiced using any suitable host cells or cell lines, including mouse or human host cells, such as airway epithelial cells, or host cell lines, such as HeLa Ohio cells, HeLa MZ cells, HeLa Kyoto cells, or A549 cells. Other suitable cell lines include a bronchial cell line called 16HBE, a tracheal cell line called THE, as well as commercially available human airway epithelial cell cultures that form well-differentiated pseudostratified mucociliary epithelia in culture (HBEpC, purchased from Promocell, Heidelberg Germany) at an air-liquid interphase (in so called ALI cultures). These cells can be used as models for HRV infection. In another embodiment a stable host cell line transformed to express a relevant or required viral entry receptor (for example, CD4 and CXCR4 for HIV-1) can be produced. The host cells can be screened using a genomic library of siRNAs previously validated for functional efficacy. In another embodiment, the genomic library of siRNAs can be obtained from a commercial source such as Qiagen.

In one embodiment HeLa cells are used as the host cells. HeLa cells allow efficient silencing by siRNA transfection. Embodiments involving the testing of influenza viruses demonstrate that single influenza viruses bind to the plasma membrane both in coated and uncoated pits. At 10 min, viruses are present in coated and uncoated small vesicles, and after 30 min many are detected in larger vesicles with an appearance consistent with endosomes. The morphology of virus entry thus resembles that observed in MDCK cells except internalization is slower. Further the trajectories of influenza viruses into and out of endosomal structures are traced using Hela cells which express Rab5-GFP, which marks the early endosomes green, and Rab7-RFP which makes late endosomes red. Further, HeLa cells are to be used to study early stages of infection, transcription, and viral protein synthesis or to screen for defects in some of the later steps such as vRNP export from the nucleus.

In another embodiment A549 cells are used as the host cells. A549 cells are especially useful in embodiments involving respiratory virus infection studies, such as the influenza virus or HRV. A549 cells are an epithelial cell line of bronchial origin that has been widely used for influenza infection studies (Ehrhardt et al. 2006). The A549 cells provide a system more similar to the host cells infected in situ during influenza disease. Additionally A549 cells offer possibilities to analyze the whole replication cycle including progeny virus release and secondary infection. Unlike MDCK cells often used in influenza studies and assays, the A549 cells are of human origin and they are easily transfected by siRNAs (Graeser 2004). In a further embodiment, two influenza viruses are tested to analyze the spread of virus and secondary infection in A549 cultures in automated high-throughput formats: 1) an avian H7N7 virus the HA of which is activated by secretase cleavage in most cell lines (Wurzer et al. 2003); and 2) a human influenza strain such as the X31/Aichi/68 and a trypsin overlay formulation that is compatible with use in 96, 384, 768, 1152, 1440, 1536, 3072 well plates, or other multiwell plate formats.

It is further contemplated that embodiments of the invention can be practiced using an automated screening platform. Wherein, the screening platform can comprise a liquid handling robot, such as a Tecan and two automated microscopes, such as the CellWorx, from Applied Precision Instruments. It is anticipated that the automated screening platform can be used to perform high-throughput experimental procedures. Further, computational and experimental efforts can be combined in parallel, to optimally adapt the siRNA assays and to set-up software for fully automated data tracking, image analysis, quantification, and statistical analysis.

In some large scale embodiments screens with siRNAs covering the entire genome of the host cell line are performed. In other embodiments screens with siRNAs covering a subset of the genome (such as at least, 600, 100, 2000, 3000, 4000, 5000, 6000, 7000, 8000, 9000, 10000, 15000, 20000, 25000, or 30000 genes) of the host cell line are performed. For example, in one possible embodiment a screen with siRNAs covering at least 7,000 genes of the human genome is performed. The RISC platform allows a 7,000-gene screen to be completed in 2-4 weeks with two different cell lines for each virus strain studied. Custom-made MatLab plug-ins are then be used to thoroughly analyze and control the quality of the datasets. MatLab plug-ins allow automatic quantification of data in the images generated, and can contain quality control algorithms that automatically discard poor quality images and determine the robustness and reproducibility of the data analysis. Once analysis is completed the results allow the identification of the host proteins involved in viral entry. The viral infectome library builds on bioinformatics tools originally generated for the analysis of cDNA microarrays, but extensively modified for use with RNAi datasets. Robust statistics of large datasets insures that the most weight is given to highly significant phenotypes. Particular phenotypes are weighted by using at least three siRNAs for each gene tested and requiring that 2 out of 3 siRNAs against a gene show similar effects.

In one embodiment, an image-based assay can be employed that is more sensitive than plate-readers, and therefore yields additional information about the cell biology behind viral infection. In these embodiments high sensitivity is desired since on average only 10-20% of cells can be infected in the unperturbed control. A low ‘base line’ is related to more efficient siRNA silencing, and to differentiate between an increase and decrease in infection. This determination provides optimal information about infection pathways.

In another embodiment involving large format siRNA screens (e.g., large gene sets that cover a subset of, or the entire host cell genome) an automated liquid handling robot, such as a Tecan, which can handle 96, 384, 768, 1152, 1440, 1536, 3072 well plates, or other multiwell plates is used. Algorithms that automatically move the data generated (9 images per well; 1,430,784 images per screen, corresponding to app. 3.8 TB) to a NAS server are be used. In further embodiments a high buffer capacity, such as 1,2,3,4,5,6,7,8,9, or 10 TB, guarantees that temporary network failures will not slowdown the analytic process. In further embodiments algorithms that continuously search these large sets of images for non-analyzed images automatically place images into the analysis queue. In some of these embodiments MatLab image analysis plug-ins are used. Further, the ‘raw’ data from the screens can be subjected to bioinformatics evaluation, to screen out false positives, which allows reconstruction of the cellular systems involved in the complex process. This will allows the definition of key target host cell proteins of the molecular machinery specific for each entry route and other infection-related processes. In another embodiment the criteria used includes strong RNAi phenotypes and wide cell-type dependency.

The methods described above have been performed with siRNAs. However other suitable molecular entities can be used such as, organic or inorganic compounds, proteins such as antibodies, or nucleic acid entities such as anti-sense RNA.

In another embodiment, the host cell proteins identified that modulate viral infection are transporters, carriers, and ion channels. In one embodiment the host cell proteins are encoded from the genes ATP6AP2, ABCC4, HTR3A, APOA1, ATP1A1, SLC35C2, ATP6V1A, ATP6V1B2, ATP6V1B2, ATP6V1C1, MCOLN3, ABCE1, SLC7A1, TAP2, and KCNB2.

### V. Transporter, Carrier, and Ion Channel Inhibitors

One aspect of the present invention includes a method of inhibiting viral infection by contacting a cell with an agent that modulates a transporter, carrier, or ion channel. This method of inhibiting viral infection can be performed in vitro by contacting virally infected cells with an agent that modulates an enzyme, or in vivo by administering to a subject infected with a virus an agent that modulates a transporter, carrier, or ion channel. In one embodiment, an agent can be an inhibitor of a transporter, carrier, or ion channel. Examples of inhibitors of transporters, carriers, or ion channels that can be used in the methods and compositions of the provided invention are described below.

A. Transporters

1. Vaculoar-ATPase Inhibitors

In one embodiment, the provided invention includes methods and compositions for inhibiting V-ATPases to treat or prevent a viral infection. An example of a V-ATPase inhibitor is bafilomycin A1, a natural macrolide. Bafilomycin binds the C subunit of V0. The structure of balifomycin A1 has been modified to generate other molecules that have increased specificity for the osteoclast V-ATPase (Farina et al; Il Farmaco 56 (2001), 113-116). For instance, the bafilomycin A1 derivative (2Z, 4E)-5-(5,6-dichloro-2-indolyl)-2-methoxy-N-[4-(2,2,6,6-tetramethyl)piperidinyl]-2,4-pentadienamide is a more potent inhibitor of bone V-ATPase compared to brain V-ATPase (Mattsson et al. 2000). (2Z,4E)-5-(5,6-dichloro-2-indolyl)-2-methoxy-N-(1,2,2,6,6-pentamethylpiperidin-4-yl)-2,4-pentadienamide (SB242784) is a derivative of bafilomycin that has shown osteoclast V-ATPase selective inhibition (Nadler et al. Bioorg Med Chem. Lett. (1998) Dec. 15; 8(24):3621-6; Yu et al, Tetrahedron Letters 39 (1998) 9347-9350). 5-(5,6-Dichloro-2-indolyl)-2-methoxy-2,4-pentadienamides are also modified forms of bafilomycin that inhibit V-ATPases (Gagliardi et al. J. Med. Chem. 1998 41, 1568-1573).

Salicylihalamide A inhibits the V0 sector of the V-ATPase (Xie et al. JBC vol 279, issue 19, 19755-1973 (2004)). 2,6-dichloro-N-[3-1(1-hydroxy-1-methyl-1ethyl)-2-methyl-7-benzofuranyl]benzamide (FR167356) is an inhibitor of osteoclast V-ATPase, and to a lesser degree, lysosomal V-ATPase (Niikura et al. British Journal of Pharmacology (2004) 142, 558-566). 2,6-dichloro-N-[3-methyl-4-(3-methyl-2-oxo-1-imidazolidinyl)-8-quinolinyl]benzamide (FR202126) has specificity for osteoclast V-ATPase inhibition (Niikura et al J. of Toxicological Sciences Vol 30, No. 4 297-304 (2005)). Concanamycin A is a V-ATPase inhibitor that binds to the C subunit of V0. Finally, N-ethylmaleimide and H362/48 are also V-ATPase inhibitors.

Examples of V-ATPase inhibitors are described in, for example, U.S. Pat. Nos. 6,787,550, 7,220,769, 6,903,117, 6,506,728, 5,858,995, and 6,008,230, and US Patent Application No. 20070248672.

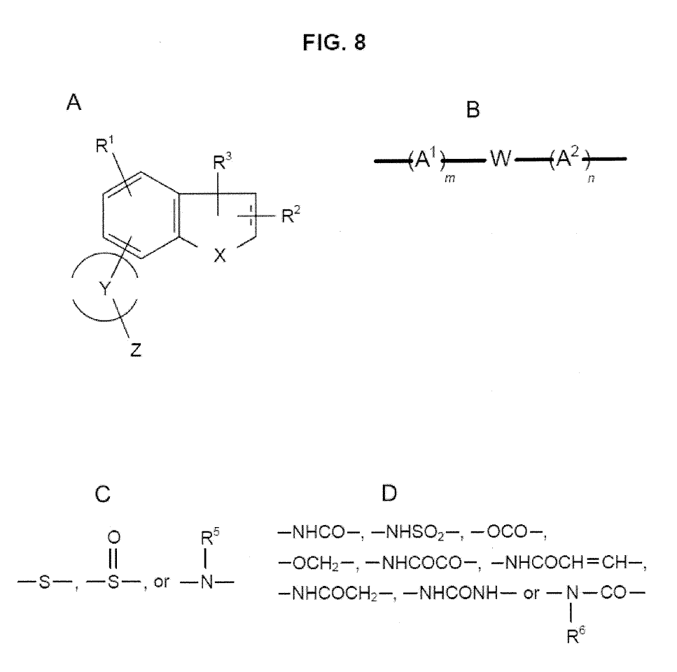

Inhibitors of V-ATPases can include the heterocyclic derivatives that can be represented by the following general formula in FIG. 8A wherein

R¹ is hydrogen, lower alkyl, an acyl group, amino, acylamino, nitro, halogen or hydroxy(lower)alkyl which can have one or more suitable substituent(s),

R² is hydrogen, lower alkyl, an acyl group, lower alkoxy, acyl(lower)alkyl, aryl, cyano, mono-(or di- or tri-)-halo (lower)alkyl, lower alkylthio or hydroxy(lower)alkyl which can have one or more suitable substituent(s),

R³ is hydrogen, lower alkyl, lower alkenyl, cyclo(lower)alkyl(lower)alkyl, halogen, an acyl group, acyl(lower)alkyl, acylamino, acylamino(lower)alkyl, acyl(lower)alkenyl, acyloxy(lower)alkyl, acyl(lower)alkylthio(lower)alkyl, amino(lower)alkyl, mono-(or di-)lower alkylamino, lower alkylthio(lower)alkyl, hydroxyimino(lower)alkyl which can have one or more suitable substituent(s), hydroxy(lower)alkyl which can have one or more suitable substituent(s), hydroxy(lower)alkylthio(lower)alkyl, cyano(lower)alkyl, mono-(or di-)lower alkoxy(lower)alkyl which can have one or more suitable substituent(s), lower alkyl substituted with aryl which can have one or more suitable substituent(s), mono-(or di-) lower alkylamino(lower)alkyl, lower alkyl substituted with heterocyclic group which can have one or more suitable substituent(s), heterocyclic group which can have one or more suitable substituent(s), heterocyclicthio, heterocyclicthio(lower), alkyl, heterocyclicoxy, heterocyclicoxy(lower)alkyl, heterocyclicaminoimino-(lower)alkyl, aryl, amino or nitro, in which R² and R³ can be linked together to form

(1) lower alkylene which can have one or more suitable substituent(s),

(2) lower alkenylene which can have one or more suitable substituent(s), or

(3) a group in FIG. 8B [wherein A¹ and A² are each lower alkylene which can have one or more suitable substituent(s) or lower alkenylene which can have one or more suitable substituent(s),

W is the formula in FIG. 8C (wherein R⁵ is hydrogen, lower alkyl or an acyl group) and m and n are each integer of 0 or 1.],

X is O or S,

Y is vinylene, or a group of the formula in FIG. 8D (wherein R⁶ is lower alkyl), Z is heterocyclic group which can have one or more suitable substituent(s), or aryl which can have one or more suitable substituent(s),

1 is an integer of 0 or 1, and [symbol] is a single bond or a double bond, and a pharmaceutically acceptable salt thereof (U.S. Pat. No. 5,858,995).

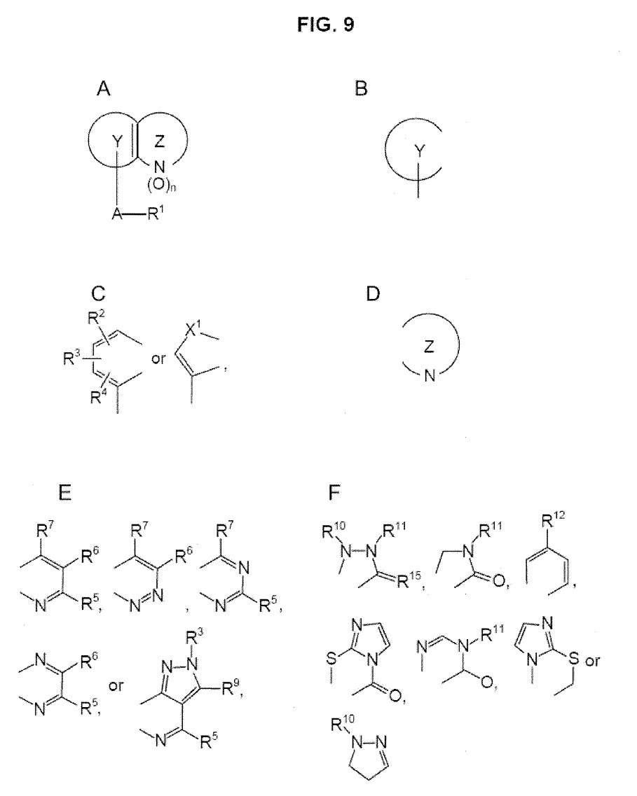

Inhibitors of V-ATPases can include compounds represented by the following general formula in FIG. 9A wherein R¹ is a heterocyclic group or aryl, each of which can be substituted with suitable substituent(s),

A is —COHN— or —NHCO—,

n is an integer of 0 or 1,

the feature in FIG. 9B is a group of the formula in FIG. 9C in which

R² is hydrogen, halogen, lower alkyl, lower alkoxy or halo (lower) alkyl,

R³ is hydrogen, halogen, lower alkyl, lower alkoxy or halo(lower)alkyl,

R⁴ is hydrogen, halogen, lower alkyl, lower alkoxy or halo(lower)alkyl, and

x¹ is O, S or NH,

the feature in FIG. 9D is a group of the formula in FIG. 9E in which

R⁵ is hydrogen or lower alkyl,

R⁸ and R⁹ are each lower alkyl,

R⁶ is hydrogen, halogen, cyano, amino, lower alkyl, substituted lower alkyl, lower alkenyl, substituted lower alkenyl, lower alkynyl, substituted lower alkynyl, lower alkylthio, lower alkylsulfinyl, lower alkylsulfonyl, a heterocyclicthio group, acyl, acylamino, aryl, substituted aryl or a heterocyclic group, and

R⁷ is hydrogen, halogen, lower alkyl, substituted lower alkyl, lower alkenyl, substituted lower alkenyl, azido, amino, substituted amino, hydrazino, substituted hydrazino, semicarbazido, substituted semicarbazido, thiosemicarbazido, substituted thiosemicarbazido, hydroxy, substituted hydroxy, mercapto, substituted mercapto, acyl or a substituted or unsubstituted heterocyclic group, or

R⁶ and R⁷ are taken together to form a group of the formula in FIG. 9F in which

R¹⁰ is hydrogen or lower alkyl,

R¹¹ is hydrogen, acyl or lower alkyl optionally substituted with a substituent selected from the group consisting of a heterocyclic group and lower alkoxy,

R¹² is hydroxy, and

R¹⁵ is O or N—R¹⁶, in which R¹⁶ is hydrogen or acyl, provided that R¹ is 2,6-dichlorophenyl when R⁶ and R⁷ are each hydrogen (U.S. Pat. No. 6,008,230).

Also reported to inhibit V-ATPases is vanadate (Chatterjee et al. PNAS 89, 6257-6261 (1992)). Tiludronate has selectivity for osteoclast V-ATPase relative to kidney V-ATPase (David et al. J. Bone Miner Res. 11(10): 1498-507 (1996)). Cadmium can directly inhibit ATPase activity of V-ATPase.

2. ATP-Binding Cassette (ABC) Transporter Inhibitors

In one embodiment, the provided invention includes methods and compositions for inhibiting ATP-binding cassette (ABC) transporters to treat or prevent a viral infection.

In one embodiment, the provided invention includes methods and compositions for inhibiting ABCC4 to treat or prevent a viral infection. The protease inhibitor 4-(2-aminoethyl)benzenesulfonyl fluoride can inhibit ABCC4 transport (Wolf C J et al. (2007) FEES J. 274:439-50). In one embodiment, the ABCC4 inhibitor can be a low molecular weight inhibitor, e.g. a small organic molecule. Examples of ABCC4 inhibitor are given in PCT Publication No. WO/2008/122666 and US Patent Application Publication No. US2006/0286041, in Reid et al. (Molecular Pharmacology, 63: 1094-1103, 2003) and in Remon et al. (J Am Soc Nephrol 13:595-603, 2002). Small organic ABCC4 inhibitors that can be used by the invention include, but are not limited to compounds selected from the group consisting of N-Acetyl-dinitrophenyl-Cysteine, Benzbromarone, Cholate, Diclofenac, Dipyrimadole, Dehydroepiandrosterone 3-glucuronide, Dehydroepiandrosterone 3-sulphate, Dilazep, Dinitrophenyl-5-glutathione, Estradiol 17-[beta]-glucuronide, Estradiol 3,17-disulphate, Estradiol 3-glucuronide, Estradiol 3-sulphate, Estrone 3-sulphate, Flurbiprofen, Folate, N5-formyl-tetrahydrofolate, Glycocholate, Glycohthocholic acid sulphate, Ibuprofen, Indomethacin, Indoprofen, Ketoprofen, Lithocholic acid sulphate, Methotrexate, MK571 ((E)-3-[[[3-[2-(7-Chloro-2-quinohnyl)ethenyl]phenyl]-[[3-dimethylamino)-3-oxopropyl]thio]methyl)thio]-propanoic acid), [alpha]-Naphthyl-[beta]-D-glucuronide, Nitrobenzyl mercaptopurine [pi]boside, Probenecid, PSC833, Sulfinpyrazone, Taurochenodeoxycholate, Taurocholate, Taurodeoxycholate, Taurohthocholate, Taurolithochohc acid sulphate, Topotecan, Trequinsin, Verapamil and Zap[pi]nast, optionally in the form of the racemates, the enantiomers, the diastereomers and optionally the pharmacologically acceptable acid addition salts and the hydrates thereof.

By acid addition salts of ABCC4 inhibitor, with pharmacologically acceptable acids are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccmate, hydrobenzoate and hydro-p-toluenesulphonate.

Other inhibitors include phosphodiesterase inhibitors, in particular structural analogs of cyclic nucleotides such as sildenafil.

In another embodiment, the provided invention includes methods and compositions for inhibiting ABCE1 to treat or prevent a viral infection. Nucleic acids that can be used to inhibit ABCE1 include, for example, ABCE1 siRNA from Santa Cruz Biotechnology, Inc. (catalog no. sc-60117) and ABCE1 shRNA Plasmid from Santa Cruz Biotechnology, Inc. (catalog no. sc-60117-SH).

In another embodiment, the provided invention includes methods and compositions for inhibiting TAP2 to treat or prevent a viral infection. Examples of nucleic acids that can be used to inhibit TAP2 include, for example, TAP2 siRNA from Santa Cruz Biotechnology, Inc. (catalog no. sc-42983) and TAP2 shRNA plasmid from Santa Cruz Biotechnology, Inc. (catalog no. sc-42983-SH).

3. Na⁺/K⁺-ATPases

In another embodiment, the provided invention includes methods and compositions for inhibiting Na⁺/K⁺-ATPases to treat or prevent a viral infection. This method of inhibiting viral infection can be performed both in vitro by contacting virally infected cells with an agent that modulates a Na⁺/K⁺-ATPase, and in vivo by administering to a subject infected with a virus an agent that modulates a Na⁺/K⁺-ATPase.

Inhibitors of Na⁺/K⁺-ATPases include cardiac glycosides. Cardiac glycosides can include, for example, digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocyrnarin, digoxin (CP 4072), strophanthidin oxime, strophanthidin semicarbazone, strophanthidinic acid lactone acetate, ernicyrnarin, sannentoside D, sarverogenin, sarmentoside A, sarmentogenin, or a pharmaceutically acceptable salt, ester, amide, or prodrug thereof.

Other Na⁺/K⁺-ATPase inhibitors are described in, for example, U.S. Pat. No. 5,240,714, which describes a non-digoxin-like Na⁺/K⁺-ATPase inhibitory factor. Those skilled in the art can also rely on screening assays to identify compounds that have Na⁺/K⁺-ATPase inhibitory activity. PCT Publications WO00/44931 and WO02/42842, for example, teach high-throughput screening assays for modulators of Na⁺/K⁺-ATPases.

Other molecules that can inhibit Na⁺/K⁺-ATPases are described in US. Patent Publication No. US20060135442 and PCT Publication No. WO2006044916.

B. Carriers

1. Solute Carrier Family Inhibitors

In another embodiment, the provided invention includes methods and compositions for inhibiting a solute carrier family member to treat or prevent a viral infection.

In another embodiment, the provided invention includes methods and compositions for inhibiting SLC35C2 to treat or prevent a viral infection. Examples of nucleic acids that can be used to inhibit SLC35C2, for example, SLC35C2 siRNA from Santa Cruz Biotechnology, Inc. (catalog no. sc-76509) and SLC35C2 shRNA Plasmid from Santa Cruz Biotechnology, Inc. (catalog no. sc-76509-SH).

In another embodiment, the provided invention includes methods and compositions for inhibiting SLC7A1 to treat or prevent a viral infection. Examples of nucleic acids that can be used to inhibit SLC7A1 include, for example, CAT-1 siRNA from Santa Cruz Biotechnology, Inc. (catalog no. sc-44923) and CAT-1 shRNA plasmid from Santa Cruz Biotechnology, Inc. (catalog no. sc-44923-SH).

2. APOA1

In another embodiment, the provided invention includes methods and compositions for inhibiting APOA1 to treat or prevent a viral infection. For example, 1, 25-(OH)2 D3 can suppress apo A1 gene expression at the transcriptional level (Wehmeier K. et la. (2005) Biochim Biophys Acta. 1737:16-26). Nucleic acids that can be used to inhibit APOA1 include, for example, apoA-I siRNA from Santa Cruz Biotechnology, Inc. (catalog no. sc-41177) and apoA-I shRNA Plasmid from Santa Cruz Biotechnology, Inc. (catalog no. sc-41177-SH).

C. Ion Channels

1.5-HT3 Receptor

In another embodiment, the provided invention includes methods and compositions for inhibiting 5-HT3 receptors to treat or prevent a viral infection. Examples of 5-HT3 antagonists that can be used in the methods and compositions described herein include, for example, cilansetron, dolasetron (Anzemet®), granisetron (Kytril®), ondansetron (Zofran®), alosetron (Lotronex®) azasetron, bemesetron (MDL-72222) cilansetron, lerisetron (F-0930-RS), lurosetron, palonosetron (Aloxi®), ramosetron (Nasea®), renzapride, tropisetron (Navoban®), zacopride, zatosetron (LY-277,359). Galanolactone is also a 5-HT3 receptor antagonist. Cisapride, renzapride, and metoclopramide possess some antagonist effect at 5-HT3 receptors. 5-HT3 receptor antagonists can prevent serotonin from binding to 5-HT3 receptors. The 5-HT3 receptor antagonist can be any other 5-HT3 receptor antagonist containing imidazole, oxazole, thiazole, pyrazole, 3-pyrroline, or pyrrolidine in its structural formula.

2. Transient Receptor Potential (TRP) Cation Channel Inhibitors

In another embodiment, the provided invention includes methods and compositions for inhibiting TRP cation channels to treat or prevent a viral infection.

In another embodiment, the provided invention includes methods and compositions for inhibiting MCOLN3 to treat or prevent a viral infection. Nucleic acids that can be used to inhibit MCOLN3 include, for example, mucolipin 3 siRNA from Santa Cruz Biotechnology, Inc. (catalog no. sc-106264) and mucolipin 3 shRNA Plasmid from Santa Cruz Biotechnology, Inc. (catalog no. sc-106264-SH).

3. Voltage-Gated Potassium Channels

In another embodiment, the provided invention includes methods and compositions for inhibiting voltage-gated potassium channels to treat or prevent a viral infection.

In another embodiment, the provided invention includes methods and compositions for inhibiting KCNB2 to treat or prevent a viral infection. Blockers of KCNB2 can include, for example, quinine, tetraethyammonium, 4-aminopyridine, and phencyclidine (reviewed in Gutman et al. (2005) Pharmacol Rev 57:473-508).

The inhibitors described herein can be suitably modified for use in the compositions and methods of the present invention.

### VI. RNA Therapeutics

Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

Double stranded RNA induced gene silencing can occur on at least three different levels: (i) transcription inactivation, which refers to RNA guided DNA or histone methylation; (ii) siRNA induced mRNA degradation; and (iii) RNA induced transcriptional attenuation. It is generally considered that the major mechanism of RNA induced silencing (RNA interference, or RNAi) in mammalian cells is mRNA degradation. RNA interference (RNAi) is a mechanism that inhibits gene expression at the stage of translation or by hindering the transcription of specific genes. Specific RNAi pathway proteins are guided by the dsRNA to the targeted messenger RNA (mRNA), where they “cleave” the target, breaking it down into smaller portions that can no longer be translated into protein. Initial attempts to use RNAi in mammalian cells focused on the use of long strands of dsRNA. However, these attempts to induce RNAi met with limited success, due in part to the induction of the interferon response, which results in a general, as opposed to a target-specific, inhibition of protein synthesis. Thus, long dsRNA is not a viable option for RNAi in mammalian systems. Another outcome is epigenetic changes to a gene—histone modification and DNA methylation—affecting the degree the gene is transcribed.

More recently it has been shown that when short (18-30 bp) RNA duplexes are introduced into mammalian cells in culture, sequence-specific inhibition of target mRNA can be realized without inducing an interferon response. Certain of these short dsRNAs, referred to as small inhibitory RNAs (“siRNAs”), can act catalytically at sub-molar concentrations to cleave greater than 95% of the target mRNA in the cell. A description of the mechanisms for siRNA activity, as well as some of its applications are described in Provost et al., Ribonuclease Activity and RNA Binding of Recombinant Human Dicer, EMBO J., 2002 Nov. 1; 21(21): 5864-5874; Tabara et al., The dsRNA Binding Protein RDE-4 Interacts with RDE-1, DCR-1 and a DexH-box Helicase to Direct RNAi in C. elegans, Cell 2002, June 28; 109(7):861-71; Ketting et al., Dicer Functions in RNA Interference and in Synthesis of Small RNA Involved in Developmental Timing in C. elegans; Martinez et al., Single-Stranded Antisense siRNAs Guide Target RNA Cleavage in RNAi, Cell 2002, Sep. 6; 110(5):563; Hutvagner & Zamore, A microRNA in a multiple-turnover RNAi enzyme complex, Science 2002, 297:2056.

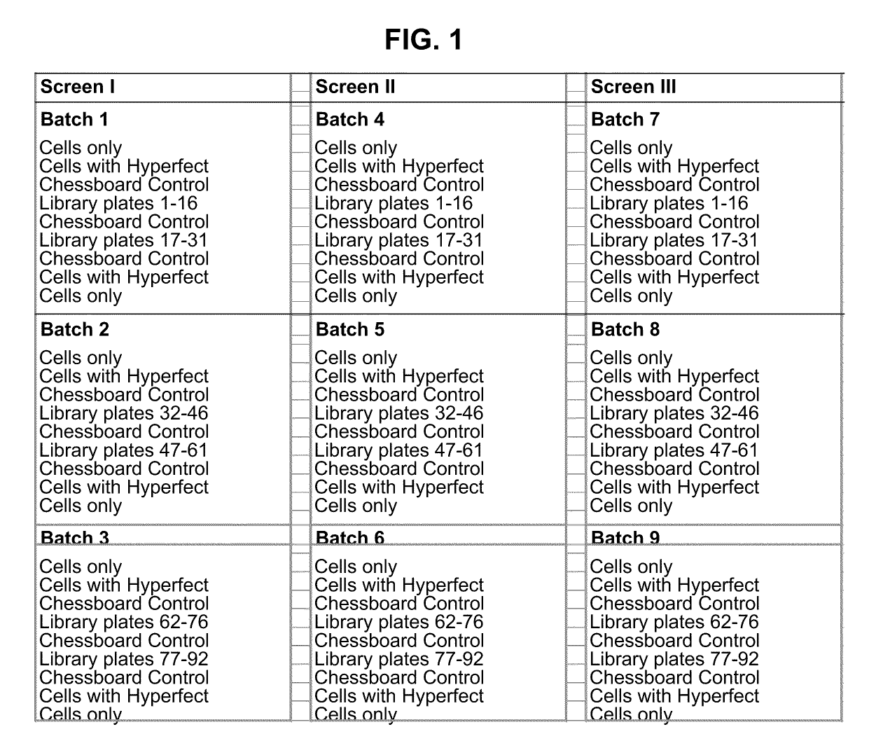

From a mechanistic perspective, introduction of long double stranded RNA into plants and invertebrate cells is broken down into siRNA by a Type III endonuclease known as Dicer. Sharp, RNA interference—2001, Genes Dev. 2001, 15:485. Dicer, a ribonuclease-III-like enzyme, processes the dsRNA into 19-23 base pair short interfering RNAs with characteristic two base 3′ overhangs. Bernstein, Caudy, Hammond, & Hannon, Role for a bidentate ribonuclease in the initiation step of RNA interference, Nature 2001, 409:363. The siRNAs are then incorporated into an RNA-induced silencing complex (RISC) where one or more helicases unwind the siRNA duplex, enabling the complementary antisense strand to guide target recognition. Nykanen, Haley, & Zamore, ATP requirements and small interfering RNA structure in the RNA interference pathway, Cell 2001, 107:309. Upon binding to the appropriate target mRNA, one or more endonucleases within the RISC cleaves the target to induce silencing. Elbashir, Lendeckel, & Tuschl, RNA interference is mediated by 21- and 22-nucleotide RNAs, Genes Dev 2001, 15:188, FIG. 1.

Generally, the antisense sequence is retained in the active RISC complex and guides the RISC to the target nucleotide sequence by means of complementary base-pairing of the antisense sequence with the target sequence for mediating sequence-specific RNA interference. It is known in the art that in some cell culture systems, certain types of unmodified siRNAs can exhibit “off target” effects. It is hypothesized that this off-target effect involves the participation of the sense sequence instead of the antisense sequence of the siRNA in the RISC complex (see for example Schwarz et al., 2003, Cell, 115, 199-208). In this instance the sense sequence is believed to direct the RISC complex to a sequence (off-target sequence) that is distinct from the intended target sequence, resulting in the inhibition of the off-target sequence In these double stranded nucleic acid molecules, each strand is complementary to a distinct target nucleic acid sequence. However, the off-targets that are affected by these dsRNAs are not entirely predictable and are non-specific.

The term “siRNA” refers to small inhibitory RNA duplexes that induce the RNA interference (RNAi) pathway. These molecules can vary in length (generally between 18-30 basepairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region. Small interfering RNA (siRNA), sometimes known as short interfering RNA or silencing RNA, are a class of 20-25 nucleotide-long double-stranded RNA molecules that play a variety of roles in biology.

While the two RNA strands do not need to be completely complementary, the strands should be sufficiently complementary to hybridize to form a duplex structure. In some instances, the complementary RNA strand can be less than 30 nucleotides, preferably less than 25 nucleotides in length, more preferably 19 to 24 nucleotides in length, more preferably 20-23 nucleotides in length, and even more preferably 22 nucleotides in length. The dsRNA of the present invention can further comprise at least one single-stranded nucleotide overhang. The dsRNA of the present invention can further comprise a substituted or chemically modified nucleotide. As discussed in detail below, the dsRNA can be synthesized by standard methods known in the art.

SiRNA can be divided into five (5) groups (non-functional, semi-functional, functional, highly functional, and hyper-functional) based on the level or degree of silencing that they induce in cultured cell lines. As used herein, these definitions are based on a set of conditions where the siRNA is transfected into said cell line at a concentration of 100 nM and the level of silencing is tested at a time of roughly 24 hours after transfection, and not exceeding 72 hours after transfection. In this context, “non-functional siRNA” are defined as those siRNA that induce less than 50% (<50%) target silencing. “Semi-functional siRNA” induce 50-79% target silencing. “Functional siRNA” are molecules that induce 80-95% gene silencing. “Highly-functional siRNA” are molecules that induce greater than 95% gene silencing. “Hyperfunctional siRNA” are a special class of molecules. For purposes of this document, hyperfunctional siRNA are defined as those molecules that: (1) induce greater than 95% silencing of a specific target when they are transfected at subnanomolar concentrations (i.e., less than one nanomolar); and/or (2) induce functional (or better) levels of silencing for greater than 96 hours. These relative functionalities (though not intended to be absolutes) can be used to compare siRNAs to a particular target for applications such as functional genomics, target identification and therapeutics.

microRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

Examples of siRNA sequences that can be used in the methods and compositions of the present inventions include those in Table 1.

TABLE 1

| Gene | OligoID | siRNA |
|---|---|---|
| ATP6AP2 | 10159_A_V3B | AAGGACTATCCTTGAGGCAAA |
| ATP6AP2 | 10159_B_V3B | ATGGGCTAATATGGATACTAA |
| ATP6AP2 | 10159_C_V3B | AACATGGATCCTGGATATGAT |
| ATP6AP2 | 10159_D_V3B | GGGAACGAGTTTAGTATATTA |
| ABCC4 | 10257_C_V3B | CTCCTAGTACTTAGAAATACA |
| ABCC4 | 10257_D_V3B | CTGGACGATCCTCTCAGTGCA |
| HTR3A | 3359_A_V3B | CAAGCTGCTATTCCACATTTA |
| HTR3A | 3359_B_V3B | TACGTGTATATTCGGCATCAA |
| APOA1 | 335_B_V3B | CGGCGCCAGACTGGCCGAGTA |
| APOA1 | 335_C_V3B | CGCTCTCGAGGAGTACACTAA |
| ATP1A1 | 476_A_V3B | CTTGATGAACTTCATCGTAAA |
| ATP1A1 | 476_B_V3B | CCCGGAAAGACTGAAAGAATA |
| ATP1A1 | 476_C_V3B | ATCCATGAAGCTGATACGACA |
| SLC35C2 | 51006_B_V3B | CTCGCTGTACACAATGACCAA |
| SLC35C2 | 51006_C_V3B | CGGCATCACCTTCTACAACAA |
| ATP6V1A | 523_A_V3B | ATGGAGGTTGATGGTAAGGTA |
| ATP6V1A | 523_B_V3B | GAGCTTGAATTTGAAGGTGTA |
| ATP6V1A | 523_C_V3B | TAAGGTAGAGTCAATTATGAA |
| ATP6V1B2 | 526_A_V3B | CTCGATTACTCAAATCCCTAT |
| ATP6V1B2 | 526_B_V3B | CACGGTTAATGAAGTCTGCTA |
| ATP6V1B2 | 526_C_V3B | ACCATGTTACCCTGTAATTAA |
| ATP6V1B2 | 526_D_V3B | GAGGATATGCTTGGTCGGGTA |
| ATP6V1C1 | 528_A_V3B | CTCGAGCATCTGCATACAATA |
| ATP6V1C1 | 528_D_V3B | AAGGGAGTAACTCAGATTGAT |
| MCOLN3 | 55283_B_V3B | ATGACTTTACTCTGACTATAA |
| MCOLN3 | 55283_D_V3B | TCCGTTGGGAATCATGCTTAT |
| ABCE1 | 6059_B_V3B | CCAGTTGGGTTCTAAATTGTA |
| ABCE1 | 6059_C_V3B | TTCCGTGGATCTGAATTACAA |
| ABCE1 | 6059_D_V3B | TGGCGCCTTATCAATTGTCAA |
| SLC7A1 | 6541_A_V3B | ACGGATCTGGATATACACTAT |
| SLC7A1 | 6541_B_V3B | ACGCTTATGACTCCTAATGTA |
| TAP2 | 6891_A_V3B | CAGGACCAGGTGAACAACAAA |
| TAP2 | 6891_D_V3B | CACCATGTCTCGAATCAACTT |
| KCNB2 | 9312_A_V3B | CTGCGGCTTTGTCCAGTTCAA |
| KCNB2 | 9312_C_V3B | CAACTCAATGACAACCGCCAA |

The siRNA sequences described herein can be suitably modified for use in the compositions and methods of the present invention.

### VII. Antibody-Based Therapeutics

The present invention embodies an agent that modulates a transporter, carrier, or ion channel. Such modulating agents include, but are not limited to, proteins, peptides, peptidomimetics, peptoids, or any other forms of a molecule, which bind to, and alter the signaling or function associated with a transporter, carrier, or ion channel, have an inhibitory or stimulatory effect on the transporter, carrier, or ion channel, or have a stimulatory or inhibitory effect on the expression or activity of the transporter, carrier, or ion channel.

In one embodiment, the present invention provides an antibody-based agent targeting a transporter, carrier, or ion channel. The antibody-based agent in any suitable form of an antibody e.g., monoclonal, polyclonal, or synthetic, can be utilized in the therapeutic methods disclosed herein. The antibody-based agents include any target-binding fragment of an antibody and also peptibodies, which are engineered therapeutic molecules that can bind to human drug targets and contain peptides linked to the constant domains of antibodies. In one embodiment, the antibodies used for targeting a transporter, carrier, or ion channel are humanized antibodies. Methods for humanizing antibodies are well known in the art. In another embodiment, the therapeutic antibodies comprise an antibody generated against a transporter, carrier, or ion channel described in the present invention, wherein the antibody is conjugated to another agent, for example, a cytotoxic agent.

The present invention also embodies the use of any pharmacologic agent that can be conjugated to an antibody or an antibody binding fragment, and delivered in active form. Examples of such agents include cytotoxins, radioisotopes, hormones such as a steroid, anti-metabolites such as cytosines, and chemotherapeutic agents. Other embodiments can include agents such as a coagulant, a cytokine, growth factor, bacterial endotoxin or a moiety of bacterial endotoxin. The targeting antibody-based agent directs the toxin to, and thereby selectively modulates the cell expressing the targeted transporter, carrier, or ion channel. In another embodiment, therapeutic antibodies employ cross-linkers that provide high in vivo stability (Thorpe et al., Cancer Res., 48:6396, 1988). In any event, it is proposed that agents such as these can, if desired, be successfully conjugated to an antibody or an antibody binding fragment, in a manner that will allow their targeting, internalization, release or presentation at the site of the targeted cells expressing the transporter, carrier, or ion channel as required using known conjugation technology.

### VIII. Transgenic Cells and Non-Human Mammals

Transgenic animal models, including recombinant and knock-out animals, can be generated from the host nucleic acids described herein. Exemplary transgenic non-human mammals include, but are not limited to, mice, rats, chickens, cows, and pigs. In certain examples, a transgenic non-human mammal has a knock-out of one or more of the target sequences associated with a transporter, carrier, or ion channel, and has a decreased viral susceptibility, for example infection by HRV or a poxvirus. Such knock-out animals are useful for studying the stages of viral infection and reducing the transmission of viruses from animals to humans. In addition, animal viruses that utilize the same targets provided herein can be analyzed in the animals.

Expression of the sequence used to knock-out or functionally delete the desired gene can be regulated by choosing the appropriate promoter sequence. For example, constitutive promoters can be used to ensure that the functionally deleted gene is never expressed by the animal. In contrast, an inducible promoter can be used to control when the transgenic animal does or does not express the gene of interest. Exemplary inducible promoters include tissue-specific promoters and promoters responsive or unresponsive to a particular stimulus (such as light, oxygen, chemical concentration), including the tetracycline/doxycycoine regulated promoters (TET-off, TET-on), ecdysone-inducible promoter, and the Cre/loxP recombinase system.

In one embodiment a transgenic mouse with a human transporter, carrier, or ion channel gene or a disrupted endogenous kinase gene, can be examined after exposure to various mammalian viruses, such as influenza or poxvirus. Comparison data can provide insight into the life cycles of the virus and related viruses. Moreover, knock-out animals (such as pigs) that are otherwise susceptible to an infection (for example HRV infection) can be made to determine the resistance to infection conferred by disruption of the gene.

In an alternative embodiment a transgenic pig with a human gene or a disrupted endogenous kinase gene, can be produced and used as an animal model to determine susceptibility to viral infections including influenza, HRV infection, or poxvirus infections. Transgenic animals, including methods of making and using transgenic animals, are described in various patents and publication, such as WO 01/43540; WO 02/19811; U.S. Pub. Nos: 2001-0044937 and 2002-0066117; and U.S. Pat. Nos. 5,859,308; 6,281,408; and 6,376,743; which are herein incorporated by reference.

### IX. Methods of Treatment

One embodiment of the present invention relates to methods of using pharmaceutical compositions and kits comprising agents that inhibit a transporter, carrier, or ion channel, or transporters, carriers, and ion channels, to inhibit or decrease a viral infection. Another embodiment of the present invention provides methods, pharmaceutical compositions, and kits for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying viral infection. Also, a therapeutic benefit is achieved with the eradication or amelioration of one or more of the physiological symptoms associated with the underlying viral infection such that an improvement is observed in the animal subject, notwithstanding the fact that the animal subject can still be afflicted with the underlying virus.

For embodiments where a prophylactic benefit is desired, a pharmaceutical composition of the invention can be administered to a patient at risk of developing viral infection such as HRV, or HIV, or to a patient reporting one or more of the physiological symptoms of a viral infection, even though a diagnosis of the condition may not have been made. Administration can prevent the viral infection from developing, or it can reduce, lessen, shorten and/or otherwise ameliorate the viral infection that develops. The pharmaceutical composition can modulate a target transporter, carrier, or ion channel activity. Wherein, the term modulate includes inhibition of a target transporter, carrier, or ion channel or alternatively activation of a transporter, carrier, or ion channel.

Reducing the activity of a transporter, carrier, or ion channel is also referred to as “inhibiting” the transporter, carrier, or ion channel. The term “inhibits” and its grammatical conjugations, such as “inhibitory,” do not require complete inhibition, but refer to a reduction in transporter, carrier, or ion channel activity. In another embodiment such reduction is by at least 50%, at least 75%, at least 90%, and can be by at least 95% of the activity of the enzyme in the absence of the inhibitory effect, e.g., in the absence of an inhibitor. Conversely, the phrase “does not inhibit” and its grammatical conjugations refer to situations where there is less than 20%, less than 10%, and can be less than 5%, of reduction in enzyme activity in the presence of the agent. Further the phrase “does not substantially inhibit” and its grammatical conjugations refer to situations where there is less than 30%, less than 20%, and in one embodiment less than 10% of reduction in enzyme activity in the presence of the agent.

Increasing the activity of a transporter, carrier, or ion channel, is also referred to as “activating” the transporter, carrier, or ion channel. The term “activated” and its grammatical conjugations, such as “activating,” do not require complete activation, but refer to an increase in transporter, carrier, or ion channel activity. In another embodiment such increase is by at least 50%, at least 75%, at least 90%, and can be by at least 95% of the activity of the enzyme in the absence of the activation effect, e.g., in the absence of an activator. Conversely, the phrase “does not activate” and its grammatical conjugations refer to situations where there is less than 20%, less than 10%, and can be less than 5%, of an increase in enzyme activity in the presence of the agent. Further the phrase “does not substantially activate” and its grammatical conjugations refer to situations where there is less than 30%, less than 20%, and in another embodiment less than 10% of an increase in enzyme activity in the presence of the agent.

The ability to reduce enzyme activity is a measure of the potency or the activity of an agent, or combination of agents, towards or against the enzyme. Potency can be measured by cell free, whole cell and/or in vivo assays in terms of IC50, Kᵢ and/or ED50 values. An IC50 value represents the concentration of an agent required to inhibit enzyme activity by half (50%) under a given set of conditions. A Kᵢ value represents the equilibrium affinity constant for the binding of an inhibiting agent to the enzyme. An ED50 value represents the dose of an agent required to effect a half-maximal response in a biological assay. Further details of these measures will be appreciated by those of ordinary skill in the art, and can be found in standard texts on biochemistry, enzymology, and the like.

The present invention also includes kits that can be used to treat viral infection. These kits comprise an agent or combination of agents that inhibits a transporter, carrier, or ion channel, or transporters, carriers, and ion channels, and optionally instructions teaching the use of the kit according to the various methods and approaches described herein. Such kits can also include information, such as scientific literature references, package insert materials, clinical trial results, and/or summaries of these and the like, which indicate or establish the activities and/or advantages of the agent. Such information can be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

### X. Formulations, Routes of Administration, and Effective Doses

Yet another aspect of the present invention relates to formulations, routes of administration and effective doses for pharmaceutical compositions comprising an agent or combination of agents of the instant invention. Such pharmaceutical compositions can be used to treat viral infections as described above.

Compounds of the invention can be administered as pharmaceutical formulations including those suitable for oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999).

In various embodiments, the pharmaceutical composition includes carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In another embodiment, the pharmaceutical preparation is substantially free of preservatives. In another embodiment, the pharmaceutical preparation can contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)). It will be recognized that, while any suitable carrier known to those of ordinary skill in the art can be employed to administer the compositions of this invention, the type of carrier will vary depending on the mode of administration.

Compounds can also be encapsulated within liposomes using well-known technology. Biodegradable microspheres can also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

The compound can be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 2.sup.87-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

The concentration of drug can be adjusted, the pH of the solution buffered and the isotonicity adjusted to be compatible with intravenous injection, as is well known in the art.

The compounds of the invention can be formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration. Suitable formulations and additional carriers are described in. Remington “The Science and Practice of Pharmacy” (20^{th} Ed., Lippincott Williams & Wilkins, Baltimore Md.), the teachings of which are incorporated by reference in their entirety herein.

The agents or their pharmaceutically acceptable salts can be provided alone or in combination with one or more other agents or with one or more other forms. For example a formulation can comprise one or more agents in particular proportions, depending on the relative potencies of each agent and the intended indication. For example, in compositions for targeting two different host targets, and where potencies are similar, about a 1:1 ratio of agents can be used. The two forms can be formulated together, in the same dosage unit e.g., in one cream, suppository, tablet, capsule, aerosol spray, or packet of powder to be dissolved in a beverage; or each form can be formulated in a separate unit, e.g., two creams, two suppositories, two tablets, two capsules, a tablet and a liquid for dissolving the tablet, two aerosol sprays, or a packet of powder and a liquid for dissolving the powder, etc.

The term “pharmaceutically acceptable salt” means those salts which retain the biological effectiveness and properties of the agents used in the present invention, and which are not biologically or otherwise undesirable. For example, a pharmaceutically acceptable salt does not interfere with the beneficial effect of an agent of the invention in inhibiting a transporter, carrier, or ion channel, such as a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide refers to those which retain biological effectiveness and properties of the agents used in the present invention, and which are not biologically or otherwise undesirable. For example, the ester or amide does not interfere with the beneficial effect of an agent of the invention in inhibiting a transporter, carrier, or ion channel, such as a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2. Typical esters include ethyl, methyl, isobutyl, ethylene glycol, and the like. Typical amides include unsubstituted amides, alkyl amides, dialkyl amides, and the like.

In another embodiment, an agent can be administered in combination with one or more other compounds, forms, and/or agents, e.g., as described above. Pharmaceutical compositions comprising combinations of a transporter, carrier, or ion channel inhibitor with one or more other active agents can be formulated to comprise certain molar ratios. For example, molar ratios of about 99:1 to about 1:99 of a transporter, carrier, or ion channel inhibitor to the other active agent can be used. In some subset of the embodiments, the range of molar ratios of transporter, carrier, or ion channel inhibitor: other active agent is selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of transporter, carrier, or ion channel inhibitor: other active agent can be about 1:9, and in another embodiment can be about 1:1. The two agents, forms and/or compounds can be formulated together, in the same dosage unit e.g., in one cream, suppository, tablet, capsule, or packet of powder to be dissolved in a beverage; or each agent, form, and/or compound can be formulated in separate units, e.g., two creams, suppositories, tablets, two capsules, a tablet and a liquid for dissolving the tablet, an aerosol spray a packet of powder and a liquid for dissolving the powder, etc.

If necessary or desirable, the agents and/or combinations of agents can be administered with still other agents. The choice of agents that can be co-administered with the agents and/or combinations of agents of the instant invention can depend, at least in part, on the condition being treated. Agents of particular use in the formulations of the present invention include, for example, any agent having a therapeutic effect for a viral infection, including, e.g., drugs used to treat inflammatory conditions. For example, in treatments for HRV, in one embodiment formulations of the instant invention can additionally contain one or more conventional anti-inflammatory drugs, such as an NSAID, e.g., ibuprofen, naproxen, acetaminophen, ketoprofen, or aspirin. In one alternative embodiment for the treatment of influenza formulations of the instant invention can additionally contain one or more conventional influenza antiviral agents, such as amantadine, rimantadine, zanamivir, and oseltamivir. In treatments for retroviral infections, such as HIV, formulations of the instant invention can additionally contain one or more conventional antiviral drug, such as protease inhibitors (lopinavir/ritonavir {Kaletra}, indinavir {Crixivan}, ritonavir {Norvir}, nelfinavir {Viracept}, saquinavir hard gel capsules {Invirase}, atazanavir {Reyataz}, amprenavir {Agenerase}, fosamprenavir {Telzir}, tipranavir{Aptivus}), reverse transcriptase inhibitors, including non-Nucleoside and Nucleoside/nucleotide inhibitors (AZT {zidovudine, Retrovir}, ddI {didanosine, Videx}, 3TC {lamivudine, Epivir}, d4T {stavudine, Zerit}, abacavir {Ziagen}, FTC {emtricitabine, Emtriva}, tenofovir {Viread}, efavirenz {Sustiva} and nevirapine {Viramune}), fusion inhibitors T20 {enfuvirtide, Fuzeon}, integrase inhibitors (MK-0518 and GS-9137), and maturation inhibitors (PA-457 {Bevirimat}). As another example, formulations can additionally contain one or more supplements, such as vitamin C, E or other anti-oxidants.

The agent(s) (or pharmaceutically acceptable salts, esters or amides thereof) can be administered per se or in the form of a pharmaceutical composition wherein the active agent(s) is in an admixture or mixture with one or more pharmaceutically acceptable carriers. A pharmaceutical composition, as used herein, can be any composition prepared for administration to a subject. Pharmaceutical compositions for use in accordance with the present invention can be formulated in conventional manner using one or more physiologically acceptable carriers, comprising excipients, diluents, and/or auxiliaries, e.g., which facilitate processing of the active agents into preparations that can be administered. Proper formulation can depend at least in part upon the route of administration chosen. The agent(s) useful in the present invention, or pharmaceutically acceptable salts, esters, or amides thereof, can be delivered to a patient using a number of routes or modes of administration, including oral, buccal, topical, rectal, transdermal, transmucosal, subcutaneous, intravenous, and intramuscular applications, as well as by inhalation.

For oral administration, the agents can be formulated readily by combining the active agent(s) with pharmaceutically acceptable carriers well known in the art. Such carriers enable the agents of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Generally, the agents of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use can contain agent(s) of this invention with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In another embodiment, oils or non-aqueous solvents can be required to bring the agents into solution, due to, for example, the presence of large lipophilic moieties. Alternatively, emulsions, suspensions, or other preparations, for example, liposomal preparations, can be used. With respect to liposomal preparations, any known methods for preparing liposomes for treatment of a condition can be used. See, for example, Bangham et al., J. Mol. Biol. 23: 238-252 (1965) and Szoka et al:, Proc. Natl. Acad. Sci. USA 75: 4194-4198 (1978), incorporated herein by reference. Ligands can also be attached to the liposomes to direct these compositions to particular sites of action. Agents of this invention can also be integrated into foodstuffs, e.g., cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked polyvinyl pyrrolidone, agar, or alginic acid or a salt thereof such as sodium alginate. The agents can also be formulated as a sustained release preparation.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents can be dissolved or suspended in suitable liquids, such as fatty oils, liquid paraffin, or liquid polyethylene glycols. In addition, stabilizers can be added. All formulations for oral administration should be in dosages suitable for administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or can contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Suitable fillers or carriers with which the compositions can be administered include agar, alcohol, fats, lactose, starch, cellulose derivatives, polysaccharides, polyvinylpyrrolidone, silica, sterile saline and the like, or mixtures thereof used in suitable amounts. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

A syrup or suspension can be made by adding the active compound to a concentrated, aqueous solution of a sugar, e.g., sucrose, to which can also be added any accessory ingredients. Such accessory ingredients can include flavoring, an agent to retard crystallization of the sugar or an agent to increase the solubility of any other ingredient, e.g., as a polyhydric alcohol, for example, glycerol or sorbitol.

When formulating compounds of the invention for oral administration, it can be desirable to utilize gastroretentive formulations to enhance absorption from the gastrointestinal (GI) tract. A formulation which is retained in the stomach for several hours can release compounds of the invention slowly and provide a sustained release that can be used in methods of the invention. Disclosure of such gastro-retentive formulations are found in Klausner, E. A.; Lavy, E.; Barta, M.; Cserepes, E.; Friedman, M.; Hoffman, A. 2003 “Novel gastroretentive dosage forms: evaluation of gastroretentivity and its effect on levodopa in humans.” Pharm. Res. 20, 1466-73, Hoffman, A.; Stepensky, D.; Lavy, E.; Eyal, S. Klausner, E.; Friedman, M. 2004 “Pharmacokinetic and pharmacodynamic aspects of gastroretentive dosage forms” Int. J. Pharm. 11, 141-53, Streubel, A.; Siepmann, J; Bodmeier, R.; 2006 “Gastroretentive drug delivery systems” Expert Opin. Drug Deliver. 3, 217-3, and Chavanpatil, M. D.; Jain, P.; Chaudhari, S.; Shear, R.; Vavia, P. R. “Novel sustained release, swellable and bioadhesive gastroretentive drug delivery system for olfoxacin” Int. J. Pharm. 2006 epub March 24. Expandable, floating and bioadhesive techniques can be utilized to maximize absorption of the compounds of the invention.

The compounds of the invention can be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible, biodegradable, such as poly(lactic-co-glycolic)acid. These materials can be made into micro or nanospheres, loaded with drug and further coated or derivatized to provide superior sustained release performance. Vehicles suitable for periocular or intraocular injection include, for example, suspensions of therapeutic agent in injection grade water, liposomes and vehicles suitable for lipophilic substances. Other vehicles for periocular or intraocular injection are well known in the art.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

When administration is by injection, the active compound can be formulated in aqueous solutions, specifically in physiologically compatible buffers such as Hanks solution, Ringer's solution, or physiological saline buffer. The solution can contain formulatory agents such as suspending, stabilizing and/or dispersing agents. Alternatively, the active compound can be in powder form for constitution with a suitable vehicle, e.g., sterile pyrogen-free water, before use. In another embodiment, the pharmaceutical composition does not comprise an adjuvant or any other substance added to enhance the immune response stimulated by the peptide. In another embodiment, the pharmaceutical composition comprises a substance that inhibits an immune response to the peptide. Methods of formulation are known in the art, for example, as disclosed in Remington's Pharmaceutical Sciences, latest edition, Mack Publishing Co., Easton P.

In addition to the formulations described previously, the agents can also be formulated as a depot preparation. Such long acting formulations can be administered by implantation or transcutaneous delivery (for example subcutaneously or intramuscularly), intramuscular injection or use of a transdermal patch. Thus, for example, the agents can be formulated with suitable polymeric or hydrophobic materials (for example as an emulsion in an acceptable oil) or ion exchange resins, or as sparingly soluble derivatives, for example, as a sparingly soluble salt.

In another embodiment, pharmaceutical compositions comprising one or more agents of the present invention exert local and regional effects when administered topically or injected at or near particular sites of infection. Direct topical application, e.g., of a viscous liquid, solution, suspension, dimethylsulfoxide (DMSO)-based solutions, liposomal formulations, gel, jelly, cream, lotion, ointment, suppository, foam, or aerosol spray, can be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations can also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In another embodiment, local/topical formulations comprising a transporter, carrier, or ion channel inhibitor are used to treat epidermal or mucosal viral infections.

Pharmaceutical compositions of the present invention can contain a cosmetically or dermatologically acceptable carrier. Such carriers are compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, an agent or combination of agents of the instant invention can be formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Ointments and creams can, for example, be formulated with an aqueous or oily base with the addition of suitable thickening and/or gelling agents. Lotions can be formulated with an aqueous or oily base and will in general also containing one or more emulsifying agents, stabilizing agents, dispersing agents, suspending agents, thickening agents, or coloring agents. The construction and use of transdermal patches for the delivery of pharmaceutical agents is well known in the art. See, e.g., U.S. Pat. Nos. 5,023,252, 4,992,445 and 5,001,139. Such patches can be constructed for continuous, pulsatile, or on demand delivery of pharmaceutical agents.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

The compositions according to the present invention can be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. These compositions can be prepared according to conventional methods. Other than the agents of the invention, the amounts of the various constituents of the compositions according to the invention are those conventionally used in the art. These compositions in particular constitute protection, treatment or care creams, milks, lotions, gels or foams for the face, for the hands, for the body and/or for the mucous membranes, or for cleansing the skin. The compositions can also consist of solid preparations constituting soaps or cleansing bars.

Compositions of the present invention can also contain adjuvants common to the cosmetic and dermatological fields, such as hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants are those conventionally used in the fields considered and, for example, are from about 0.01% to about 20% of the total weight of the composition. Depending on their nature, these adjuvants can be introduced into the fatty phase, into the aqueous phase and/or into the lipid vesicles.

In another embodiment, ocular viral infections can be effectively treated with ophthalmic solutions, suspensions, ointments or inserts comprising an agent or combination of agents of the present invention. Eye drops can be prepared by dissolving the active ingredient in a sterile aqueous solution such as physiological saline, buffering solution, etc., or by combining powder compositions to be dissolved before use. Other vehicles can be chosen, as is known in the art, including but not limited to: balance salt solution, saline solution, water soluble polyethers such as polyethyene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

The solubility of the components of the present compositions can be enhanced by a surfactant or other appropriate co-solvent in the composition. Such cosolvents include polysorbate 20, 60, and 80, Pluronic F68, F-84 and P-103, cyclodextrin, or other agents known to those skilled in the art. Such co-solvents can be employed at a level of from about 0.01% to 2% by weight.

The compositions of the invention can be packaged in multidose form. Preservatives can be preferred to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In the prior art ophthalmic products, such preservatives can be employed at a level of from 0.004% to 0.02%. In the compositions of the present application the preservative, preferably benzalkonium chloride, can be employed at a level of from 0.001% to less than 0.01%, e.g. from 0.001% to 0.008%, preferably about 0.005% by weight. It has been found that a concentration of benzalkonium chloride of 0.005% can be sufficient to preserve the compositions of the present invention from microbial attack.

In another embodiment, viral infections of the ear can be effectively treated with otic solutions, suspensions, ointments or inserts comprising an agent or combination of agents of the present invention.

In another embodiment, the agents of the present invention are delivered in soluble rather than suspension form, which allows for more rapid and quantitative absorption to the sites of action. In general, formulations such as jellies, creams, lotions, suppositories and ointments can provide an area with more extended exposure to the agents of the present invention, while formulations in solution, e.g., sprays, provide more immediate, short-term exposure.

In another embodiment relating to topical/local application, the pharmaceutical compositions can include one or more penetration enhancers. For example, the formulations can comprise suitable solid or gel phase carriers or excipients that increase penetration or help delivery of agents or combinations of agents of the invention across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, α-bisabolol, d-limonene, urea and N,N-diethyl-m-toluamide, and the like. Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In another embodiment, the pharmaceutical compositions will include one or more such penetration enhancers.

In another embodiment, the pharmaceutical compositions for local/topical application can include one or more antimicrobial preservatives such as quaternary ammonium compounds, organic mercurials, p-hydroxy benzoates, aromatic alcohols, chlorobutanol, and the like.

Gastrointestinal viral infections can be effectively treated with orally- or rectally delivered solutions, suspensions, ointments, enemas and/or suppositories comprising an agent or combination of agents of the present invention.

Respiratory viral infections can be effectively treated with aerosol solutions, suspensions or dry powders comprising an agent or combination of agents of the present invention. Administration by inhalation is particularly useful in treating viral infections of the lung, such as an HRV infection. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. For example, an aerosol formulation comprising a transporter, carrier, or ion channel inhibitor can be dissolved, suspended or emulsified in a propellant or a mixture of solvent and propellant, e.g., for administration as a nasal spray or inhalant. Aerosol formulations can contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the agent or combination of agents of the present invention is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants useful in the present invention include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as the ethers. An aerosol formulation of the invention can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present invention can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of an agent of the invention such as a transporter, carrier, or ion channel inhibitor in (substantially) pure propellant or as a mixture of propellant and solvent. The solvent can be used to dissolve the agent and/or retard the evaporation of the propellant. Solvents useful in the invention include, for example, water, ethanol and glycols. Any combination of suitable solvents can be use, optionally combined with preservatives, antioxidants, and/or other aerosol components.

An aerosol formulation can also be a dispersion or suspension. A suspension aerosol formulation can comprise a suspension of an agent or combination of agents of the instant invention, e.g., a transporter, carrier, or ion channel inhibitor, and a dispersing agent. Dispersing agents useful in the invention include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as an agent or combination of agents of the invention, e.g., a transporter, carrier, or ion channel. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

The compounds of the invention can be formulated for administration as suppositories. A low melting wax, such as a mixture of triglycerides, fatty acid glycerides, Witepsol S55 (trademark of Dynamite Nobel Chemical, Germany), or cocoa butter is first melted and the active component is dispersed homogeneously, for example, by stirring. The molten homogeneous mixture is then poured into convenient sized molds, allowed to cool, and to solidify.

The compounds of the invention can be formulated for vaginal administration. Pessaries, tampons, creams, gels, pastes, foams or sprays containing in addition to the active ingredient such carriers as are known in the art to be appropriate.

It is envisioned additionally, that the compounds of the invention can be attached releasably to biocompatible polymers for use in sustained release formulations on, in or attached to inserts for topical, intraocular, periocular, or systemic administration. The controlled release from a biocompatible polymer can be utilized with a water soluble polymer to form a instillable formulation, as well. The controlled release from a biocompatible polymer, such as for example, PLGA microspheres or nanospheres, can be utilized in a formulation suitable for intra ocular implantation or injection for sustained release administration, as well. Any suitable biodegradable and biocompatible polymer can be used.

Pharmaceutical compositions suitable for use in the present invention include compositions wherein the active ingredients are present in an effective amount, i.e., in an amount effective to achieve therapeutic and/or prophylactic benefit in a host with at least one viral infection. The actual amount effective for a particular application will depend on the condition or conditions being treated, the condition of the subject, the formulation, and the route of administration, as well as other factors known to those of skill in the art. Determination of an effective amount of a transporter, carrier, or ion channel inhibitor is well within the capabilities of those skilled in the art, in light of the disclosure herein, and will be determined using routine optimization techniques.

The effective amount for use in humans can be determined from animal models. For example, a dose for humans can be formulated to achieve circulating, liver, topical and/or gastrointestinal concentrations that have been found to be effective in animals. One skilled in the art can determine the effective amount for human use, especially in light of the animal model experimental data described herein. Based on animal data, and other types of similar data, those skilled in the art can determine the effective amounts of compositions of the present invention appropriate for humans.

The effective amount when referring to an agent or combination of agents of the invention will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

Further, appropriate doses for a transporter, carrier, or ion channel inhibitor can be determined based on in vitro experimental results. For example, the in vitro potency of an agent in inhibiting a transporter, carrier, or ion channel component, such as ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2, provides information useful in the development of effective in vivo dosages to achieve similar biological effects.

In another embodiment, administration of agents of the present invention can be intermittent, for example administration once every two days, every three days, every five days, once a week, once or twice a month, and the like. In another embodiment, the amount, forms, and/or amounts of the different forms can be varied at different times of administration.

A person of skill in the art would be able to monitor in a patient the effect of administration of a particular agent. For example, HIV viral load levels can be determined by techniques standard in the art, such as measuring CD4 cell counts, and/or viral levels as detected by PCR. Other techniques would be apparent to one of skill in the art.

### EXAMPLES

### Example 1

### HRV RNAi Screening Infection Protocol

This protocol applies to 384 well plates and details the setup of the HRV assay on the Freedom EVO. All steps, unless otherwise stated, were done on the robot.

The QIAGEN druggable genome library version 3 was used. This library contains siRNAs for about 7000 genes and each gene is represented by 4 different siRNAs (total of about 28,000 siRNAs). The final siRNA concentration in each well was 30 nM, with the exception of the Eg5 control siRNA which was 5 nM. The screen was performed in triplicate (see FIG. 1 for the screen structure).

Reagents and materials for the screen included 384 well optical bottom plates (Matrix Screenmate 384 well plate black P/N 4332). Plates were labelled with barcodes prior to starting the experiment. Reagents and materials also included QIAGEN druggable genome library plates (MasterPlates), Growth medium (see below ‘Buffers and Media’), Infection medium (see below ‘Buffers and Media’), Optimem (Gibco, P/N 31985), HiPerFect Transfection Reagent (Qiagen, #1034452), siRNA Dilution Buffer (see below ‘Buffers and Media’), HeLa Ohio Cells, HRV virus, and HRV monoclonal antibody R16-7 as described in Mosser et al. J. Infectious Diseases 185, p 734, 2002. Controls used were: Control 1: anti-HRV siRNA; Control 2: ICAM siRNA; and Control 3: PAX siRNA.

Buffers and media included Growth medium (DMEM 10% FCS 1% NEAA 1% Glu; DMEM (Sigma, #D5796)+50 ml FCS, +5 ml L-Alanyl-L-Glutamine (Sigma, #G8541), +5 ml non-essential amino acid solution (Sigma, #M7145)); Infection medium (DMEM 2% FCS 30 mM MgCl₂; DMEM (Sigma, #D5796)+10 ml FCS+15 ml Magnesium chloride standard solution (Fluka, #63020, 1M)); and siRNA Dilution Buffer (QIAGEN) (100 mM KAc, 30 mM Hepes, 2 mM MgAc; Potassium acetate solution (Fluka, #60038), Magnesium acetate solution (Fluka, #63052), Hepes buffer (Sigma, #H0887), Nuclease free water (Qiagen, #1039480)).

### Generating Chessboard Control CellPlates

For a detailed description of the process, refer to Example 6. For HRV screening, chessboard control siRNAs (Scrambled, Allstars Neg, Eg5, HRV, ICAM and PAX) were diluted to a final concentration of 1.2 μM. The following volumes were required for 80 μl per well (for 1 plate): Scrambled (3100 μl); All Stars Negative (3100 μl); HRV (6000 μl); Eg5 (6000 μl); ICAM (522 μl); PAX (5700 μl); Mock (Dilution Buffer; 6000 μl). This plate was the “master chessboard plate” that was used to generate the “daughter chessboard cell plates” used in the screen.

“Cells Only” and “Cells+Hyperfect” Control CellPlates were produced as described in Example 3 and stored at −80° C. until used.

The screen structure is illustrated in FIG. 1.

### Transfection and Infection Protocol

On Day 1, transfection was performed. 30 (or 31) cell plates from the respective batch plus 3 chessboard control plates, 2 “Cell only” and 2 “Cell+Hyperfect” plates (per batch of cell plates being processed) were thawed. The 37° C. and 4° C. waterbaths was switched on and allowed to warm up or cool down, respectively. Once plates thawed, they were centrifuged at 1000 rpm for 1 minute. Plates were re-lided and loaded into the StoreX4° C. from tower 1 onwards. Towers were scanned in order to be able to run the processes below in the “specified by barcode” mode. Cell culture was prepared in a spinner flask to give 1.4 L of medium. Cell count was 15,000 cells/ml (900 cells per well). EVO was loaded and primed. 100 ml Hyperfect/Optimem (95 ml Optimem+5 ml Hyperfect) was prepared, mixed, and added to the cooled 4° C. trough. The import files were prepared so that the batch was processed as follows: Cell+Hyperfect Plate, Chessboard 1, Library plates 1−16, Chessboard 2, Library Plates 17−30/31, Chessboard 3, Cell+Hyperfect plate. The process ‘Cells_Aliquoting_Transfection’ was run with the process ‘Cells_Aliquoting’ before and after it. The “specified by barcode” mode was used. The volume of cells being added was 60 μl. Example 5 has details on the process. Incubation was for 72 hrs at 37° C. and 5% CO₂ before proceeding with infection.

On Day 4, the cells were infected. Towers were scanned in order to be able to run the process below in the “specified by barcode” mode. The 37° C. waterbath was allowed to warm up. HRV virus was added to the cooled trough (used the inlay with column 1 separated) by adding 435 μl virus to 174 ml Infection Medium. The process ‘RNAi_Infection’ was started for 17 plates and steps 3 and 4 were repeated for the remaining 17 (or 16) plates from the batch. This process consisted of the following steps: 1) removal of Growth medium, 2) addition of Infection medium (40 μl), and 3) addition of 10 μl virus in Infection Medium. Example 4 contains details on the infection process. Plates were incubated in the StoreX37° C. for 3 hrs. During this time, the PowerWasher384 was rinsed with MilliQ grade water and it was verified that all pins were working. 1.4 L Infection medium was added to a spinnerflask and the multidrop was primed.

The process ‘RNAi_Infection_VirusRemoval’ was run for all 34 (or 33) plates. Incubation was for a further 5½ hours before the plates were fixed using process ‘Fix_{—}384_Multidrop’ (Example 2 contains details).

### HRV R16-7 MAb Staining in 384 Well Plates (Manual)

This protocol applies to 384 well plates. Monoclonal anti-mouse IgG anti-RT6 16-7 (stock: 0.7 mg/ml, glycerol stock) was used. Buffers and solutions included: permeabilization buffer (0.2% Triton-X-100 (Sigma, #234729)/PBS), wash buffer (PBS/25 mM NH₄Cl), blocking buffer (1% BSA in PBS), primary antibody solution (1/15,000 dilution in blocking buffer), secondary antibody solution (polyclonal goat anti-mouse IgG (H+L) Alexa Fluor 488-labelled (Invitrogen, # A11029) 1/1000 diluted in blocking buffer and Heochst (Invitrogen, #33258) 1/10,000 diluted in blocking buffer).

The steps of the staining protocol (manual) included: 1) wash 2× with Wash Buffer, 50 μl/well, 2) permeabilize in 50 μl/well permeabilization buffer for 5 min at RT; 3) wash 2× with PBS; 4) block with 50 μl/well blocking buffer for 30 min at RT; 5) incubate with 30 μl/well primary antibody solution for 1 hr at RT (or up to 5 hrs, at 4° C.); 6) wash 3× with 70 μl/well PBS; 7) incubate with 30 μl/well secondary antibody solution for 1 hr at RT in the dark (or up to 5 hrs at 4 C); 8) wash 3× with 70 μl/well PBS; and 9) store with 70 μl/well PBS+1% Penicillin/Streptomycin (Pen/Strep, Gibco) at 4° C. in the dark.

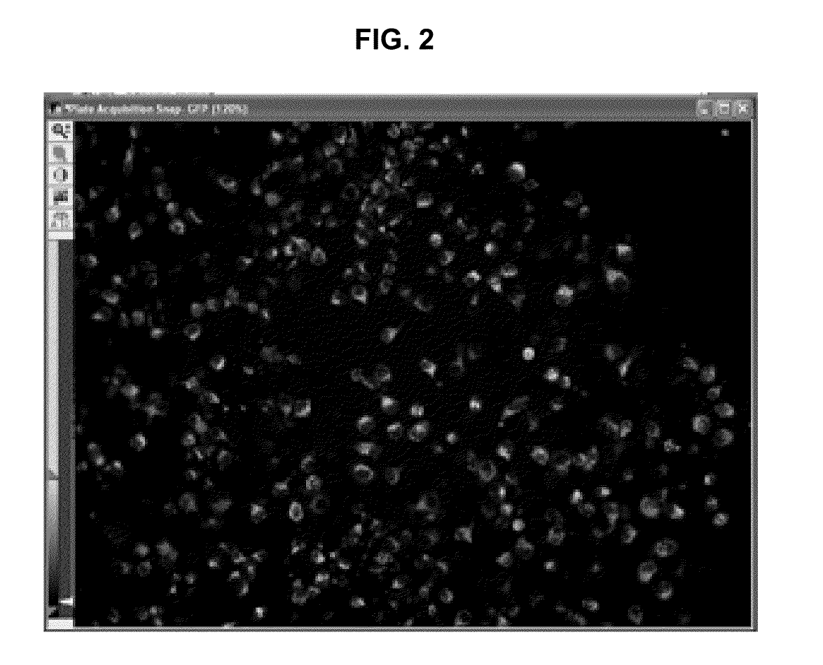

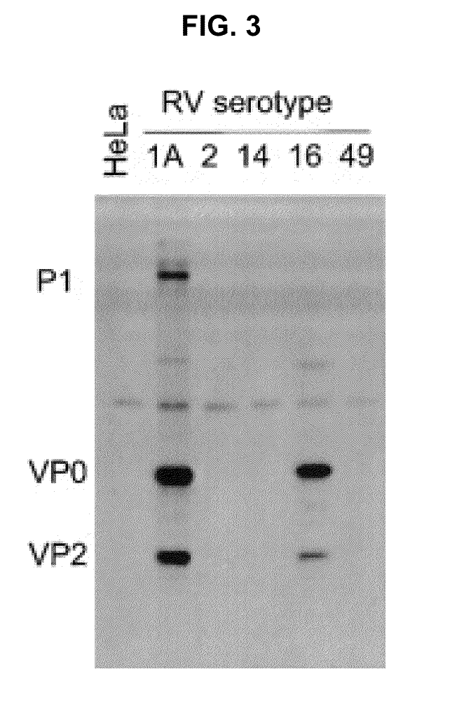

FIG. 2 illustrates a typical R16-7 HRV staining pattern. FIG. 3 shows a Western blot of lysates of HeLa cells infected with rhinovirus (RV) serotypes 1A, 2, 14, 16, or 49 or mock-infected and blotted with monoclonal antibody R16-7. The antibody reacted with the viral capsid protein VP2 and with the VP2 precursors VP0 and P1, of both RV1A and RV16, as described in Mosser et al. J. Infectious Diseases 185, p 734, 2002.

Statistical Analysis of siRNA Screening

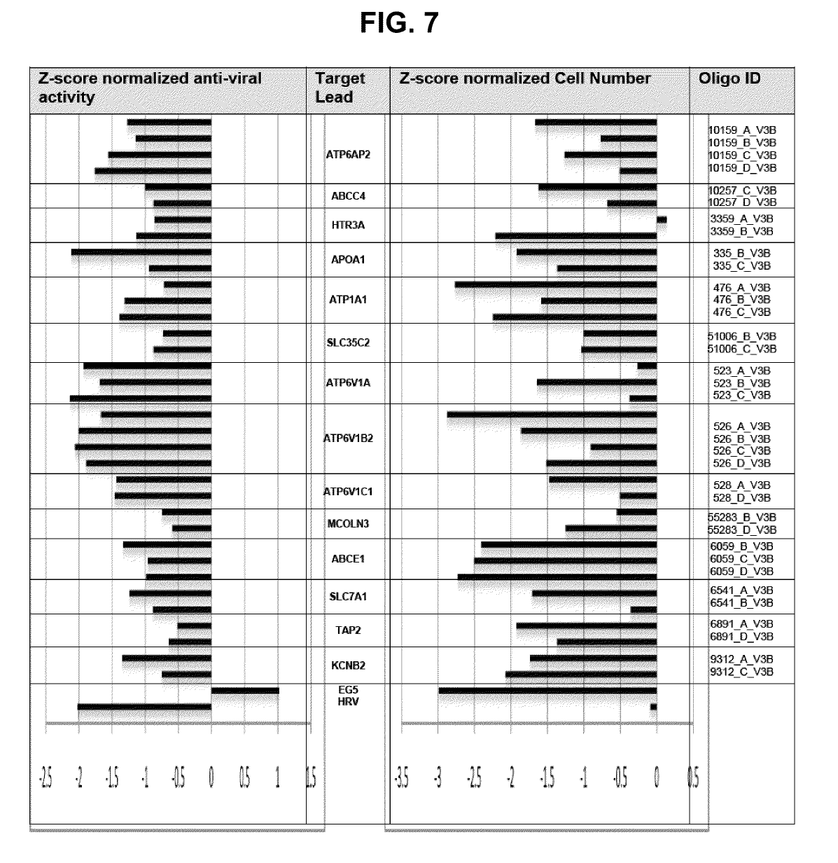

All 330 screening plates were analyzed by standard statistical methods following a similar approach as described by Boutros et al. Analysis of cell-based RNAi screens Genome Biol (2006) vol. 7 (7) pp. R66. Infection and cellcount data were analyzed separately by applying the same approach. The number of infected cells was determined with in house custom made software. Values for percent infected cells and total cell numbers were normalized plate based by a z-score algorithm but also by taking into account the respective positive and negative controls. To relate the original screen with the technical reproduction, a non coding siRNA (“scrambled”) was introduced as a reference point which neither showed an effect on infection levels nor cell numbers. Infection index and cell count represent the normalized z-scores. Hit leads were selected on the basis of not showing massive toxicity (based on WST, BrdU and cell numbers) and resulting in at least 30 percent reduction in the viral signal. FIG. 7 illustrates results of an HRV infection study using siRNA against transporters, carriers, and ion channels.

### Example 2

### Fixing Plates on the Tecan EVO (Tecan Robot Process)

This Example describes the various fixation procedures for plates on the Tecan EVO. The process was run under EVOware PLUS. There are 4 fixation processes, which differ depending on the plate or assay used. 384 well plates were loaded into the StoreX 37° C. in towers 1 to 9. 96 well plates were loaded into tower 10.

### Fix 96 LiHa

Prior to starting the process, the script is checked to ensure the amount of formaldehyde added is correct. This process adds 100 μl of formaldehyde per well of an entire 96 well plate. A formaldehyde solution is prepared which, when diluted, has a final concentration of 4% in the well. The maximum volume that is loaded is up to 100 ml, which defines the maximum number of plates that can be fixed with a certain volume/well. A 100 ml trough is added to position 1 of the cooled carrier and is filled with formaldehyde solution. 200 ul tips are used. Sufficient tips are loaded. The process is started.

### Fix 384 LiHa

Prior to starting the process, the script is checked to ensure the amount of formaldehyde added is correct. This process adds 10 μl of formaldehyde per well of an entire 384 well plate. A formaldehyde solution is prepared which, when diluted, has a final concentration of 4% in the well. The maximum volume that is loaded is up to 100 ml, which defines the maximum number of plates that can be fixed with a certain volume/well. A 100 ml trough is added to position 1 of the cooled carrier and is filled with formaldehyde solution. 200 ul tips are used. Sufficient tips are loaded. The process is started.

### Fix 384 Multidrop

The ‘fixation’ multidrop tubing was installed and flushed with 70% EtOH and water. A formaldehyde solution was prepared which when diluted had a final concentration of 4% in the well. An additional 200 ml was added as dead volume. The formaldehyde was added to a sterile beaker and the tubing was immersed into the formaldehyde and the aluminium foil was fixed over the beaker to ensure that the beaker was sealed and that the tubing reached the bottom of the beaker. If more than 500 ml formaldehyde was needed, a spinner flask was used and the “Antibody” multidrop tubing was used. The default amount of formaldehyde added per well was 10 μl, and the amount was adjusted to meet requirements. The process was started.

### Fix Compound Assay

The ‘fixation’ multidrop tubing is installed and flushed with 70% EtOH and water. A formaldehyde solution is prepared which when diluted has a final concentration of 4% in the well. An additional 200 ml is added as dead volume. The formaldehyde is added to a sterile beaker and the tubing is immersed into the formaldehyde and the aluminium foil is fixed over the beaker to ensure that the beaker is sealed and that the tubing reaches the bottom of the beaker. If more than 500 ml formaldehyde is needed, a spinner flask is used and the “Antibody” multidrop tubing is used. The default amount of formaldehyde added is 50u1, and the amount is adjusted if required to meet requirements. It is ensured that there is free space in the storex 4° C., particularly tower 1. The rinse bottle is filled on the PowerWasher384 with MilliQ water and the PowerWasher384 is primed. The PowerWasher384 is tested by dispensing and then aspirating from the rinse channel. It is ensured that all wells are clear of blockages. It is ensured that there is enough space in the powerwasher waste for the run. The process is started. Once the run has finished, the rinse channel is primed as soon as possible and, if possible, an overnight rinse is performed. Alternatively, the process “_System_PW384_Rinse Night” is run.

### Example 3

### Seeding of Cell Plates for RNAi Screens (Tecan Robot Process)

This Example describes the preparation of CellONLY and CellHyperONLY control plates in preparation for RNAi screens. Process “RNAi_Generate_Cell_ONLY” aliquoted 20 μl of Optimem into 384 well plates and process “RNAi_Generate_Cell_HyperONLY” aliquoted 15 μl Optimem. The process was run under EVOware PLUS. Plates were sealed and stored at −80° C. until used in a screen. Plates from process “RNAi_Generate_Cell_ONLY” were seeded with cells using the process “Cells_Aliquoting” and plates from process “RNAi_Generate_Cell_HyperONLY” were seeded with cells using process “Cells_Aliquoting_Transfection”.

### Example 4

### Infection for RNAi Screens

This Example describes the processes used for infection and virus removal after infection. The process was run under EVOware PLUS. Process “RNAi_Infection” consisted of 3 steps: 1) removal of Growth Medium using the PowerWasher384, 2) addition of 40 μl Infection medium with the Multidrop, and 3) addition of 10 μl virus in Infection medium with the TeMO. The PowerWasher384 was rinsed immediately after using it. The cooling for the virus trough and the heating for the medium trough were switched on.

### Example 5

### Seeding of Cell Plates for RNAi Screens (Transfections) (Tecan Robot Process)

This Example describes the seeding of plates with cells in preparation for RNAi screens (Transfection). The process was run under EVOware PLUS. The waterbath was switched on and the start button was pressed. It was ensured that the waterbath heated up before the experiment was started. Plates were seeded 72 hours prior to infection to allow suppression of translation. Cell plates (containing prealiquotted siRNAs at the desired concentration) were removed from −80° C. and left to defrost. Once thawed, the plates were centrifuged and re-lided. Plates were loaded starting at tower 1 of the StoreX 4° C. Multidrop tubing was installed and flushed with 70% EtOH and sterile distilled water. The volume dispensed per well was 60 μl. HeLa Ohio, 900 cells per well (HRV screening) were used. An appropriate amount of HiPerFect was added to the 4° C. trough. 2 ml per plate plus 25 ml per batch as dead volume was calculated. When processing a batch, the process “Cell_Aliquoting” was run before and after running the process “Cell_Aliquoting_Transfection” to seed cells onto the control ‘Cells only’ plates.

### Example 6

### Chessboard Plate Generation for siRNA Screening (Tecan Robot Process)

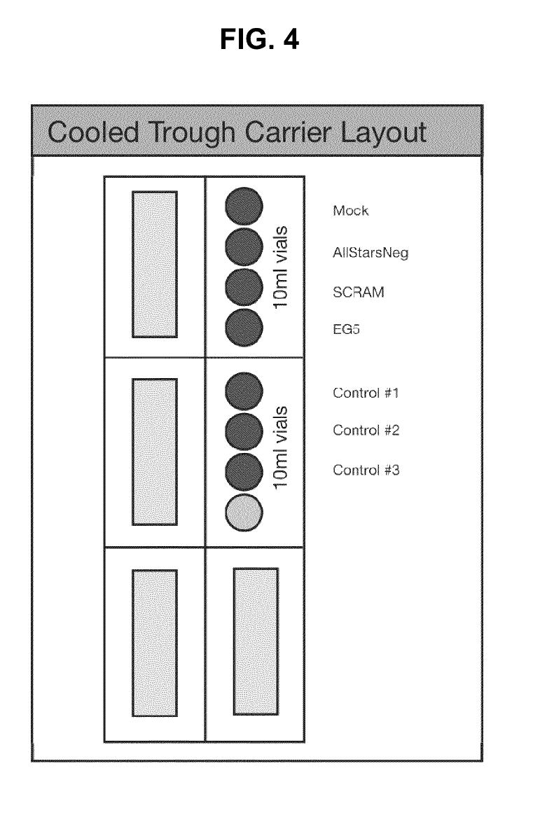

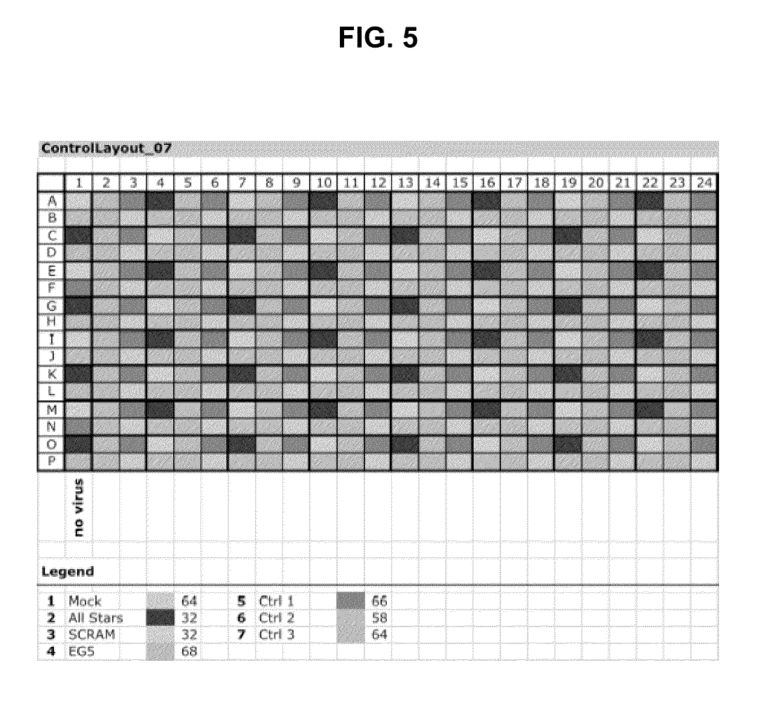

The process “RNAi_ChessboardPlate_VarVol_{—}7siRNAs” is a generic process for the Tecan robot that runs under EVOware PLUS. This process produces a Chessboard Plate at the DP level. Chessboard CPs were generated after using one of the processes “RNAi_CPfromDP_{—}3CPsfrom1DP” “RNAi_CPfromDP_{—}4CPsfrom1DP” or “RNAi_CPfromDP_{—}12CPsfrom1DP”. Hence, the concentration of siRNAs provided was 40× the final concentration. The maximum number of different siRNAs was 7. It was generally used with 4 controls (mock, AllStarsNeg, Scram and Eg5) and 3 positive control siRNAs (see FIG. 4 for cooled trough carrier layout). The positions of the siRNAs in the 4° C. trough (from top to bottom) correspond to the order (1-7). Sites 4 and 5 were used in the 4° C. trough. The maximum volume pipetted was 40 ul. FIG. 5 illustrates a control layout.

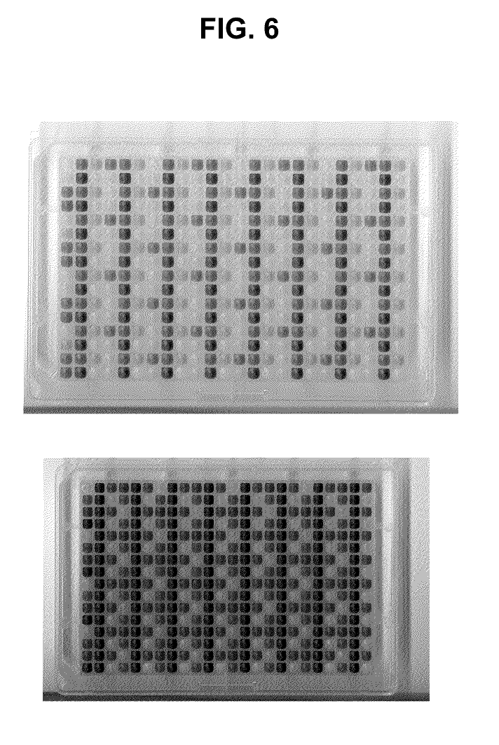

Prior to starting the process, the volume was specified inside the pipetting script by specifying the PipVolDisp variable. The number of tips consumed was 48 (200 ul conductive tips). The duration was about 20 min, depending on the volume. The labware for source siRNAs were 10 ml vials (BD) in the cooling trough inserts. The labware for plates was “384 well Matrix Round Bottom LiHA”. The liquid class was “siRNA transfer DiTi 10 ml tubes” which used liquid level detection. 35 ul of siRNAs were aliquoted for producing 9 CPs (repeated 3× for the 27 Chessboard CPs needed for a whole screen). FIG. 6 shows pictures of plates pipetted with 20 (top) and 40 ul aliquots (bottom).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A method of preventing or treating a viral infection comprising administering to a subject in need thereof an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

2. The method of claim 1 wherein the method comprises preventing a viral infection by administering to a subject in need thereof an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

3. The method of claim 1 wherein the method comprises treating a viral infection by administering to a subject in need thereof an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

4. The method of claim 1 wherein the infection is a respiratory infection.

5. The method of claim 1 wherein the virus is a respiratory virus.

6. The method of claim 1 wherein the virus is a human rhinovirus.

7. The method of claim 1 wherein the subject is a human.

8. The method of claim 1 wherein the agent is an RNA, an antibody-based agent, or a small molecule.

9. A method of preventing or treating human rhinovirus infection comprising administering to a subject in need thereof an agent that modulates a transporter, carrier, ion channel.

10. The method of claim 9 wherein the transporter is a V-ATPase, ATP-binding cassette (ABC) transporter, or Na⁺/K⁺-ATPase.

11. The method of claim 9 wherein the ion channel is a transient receptor potential (TRP) cation channel, voltage-gated potassium channel, or 5HT3-receptor.

12. The method of claim 9 wherein the carrier is a solute family carrier or APOA1.

13. The method of claim 9 wherein the method comprises preventing human rhinovirus infection by administering to a subject in need thereof an agent that modulates a transporter, carrier, or ion channel.

14. The method of claim 9 wherein the method comprises treating human rhinovirus infection by administering to a subject in need thereof an agent that modulates a transporter, carrier, or ion channel.

15. The method of claim 9 wherein the subject is a human.

16. The method of claim 9 wherein the agent in an RNA, an antibody-based agent, or a small molecule.

17. The method of claim 9 wherein the agent is a transporter, carrier, or ion channel inhibitor.

18. A method of inhibiting infection of a cell by a virus comprising contacting the cell with an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

19. The method of claim 18 wherein the method of inhibiting viral infection is performed in vitro.

20. The method of claim 18 wherein the method of inhibiting viral infection is performed in vivo.

21. The method of claim 18 wherein the infection is a respiratory infection.

22. The method of claim 18 wherein the virus is a human rhinovirus.

23. The method of claim 18 wherein the subject is a human.

24. The method of claim 18 wherein the agent is an RNA, an antibody-based agent, or a small molecule.

25. The method of claim 18 wherein the agent is a transporter, carrier, or ion channel inhibitor.

26. A method of inhibiting infection of a cell by a human rhinovirus comprising contacting the cell with an agent that modulates a transporter, carrier, or ion channel.

27. The method of claim 26 wherein the method of inhibiting viral infection is performed in vitro.

28. The method of claim 26 wherein the method of inhibiting viral infection is performed in vivo.

29. The method of claim 26 wherein the subject is a human.

30. The method of claim 26 wherein the agent is an RNA, an antibody-based agent, or a small molecule.

31. The method of claim 26 wherein the transporter, carrier, or ion channel is selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.

32. A method comprising:
(a) contacting a cell with an agent that modulates a transporter, carrier, or ion channel selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2 with a virus, and
(b) determining whether the agent inhibits infection of the cell by the virus.

33. The method of claim 32 wherein the infection is a respiratory infection.

34. The method of claim 32 wherein the virus is a human rhinovirus.

35. The method of claim 32 wherein the subject is a human.

36. The method of claim 32 wherein the agent is an RNA, an antibody-based agent, or a small molecule.

37. A method comprising:
(a) contacting a cell with an agent that modulates a transporter, carrier, or ion channel with a human rhinovirus, and
(b) determining whether the agent inhibits infection of the cell by the human rhinovirus.

38. The method of claim 37 wherein the subject is a human.

39. The method of claim 37 wherein the agent is an RNA, an antibody-based agent, or a small molecule.

40. The method of claim 37 wherein the agent is a transporter, carrier, or ion channel inhibitor.

41. The method of claim 37 wherein the transporter, carrier, or ion channel is selected from the group consisting of ATP6AP2, ABCC4, HTR3A, APOA1, ATP1A1, SLC35C2, ATP6V1A, ATP6V1B2, ATP6V1C1, MCOLN3, ABCE1, SLC7A1, TAP2, and KCNB2.
